(19) United States
(12) Patent Application Publication
(10) Pub. No.: US 2025/0345174 A1
(43) Pub. Date: Nov. 13, 2025

(54) PROSTHETIC MEDICAL DEVICE DELIVERY SYSTEM

(71) Applicant: EDWARDS LIFESCIENCES CORPORATION, Irvine, CA (US)

(72) Inventors: Kevin Gantz, Laguna Beach, CA (US); Timothy Lee Humphrey, Anaheim, CA (US); Kurt Kelly Reed, Costa Mesa, CA (US); Darshin S. Patel, Lake Forest, CA (US)

(21) Appl. No.: 19/274,914

(22) Filed: Jul. 21, 2025

Related U.S. Application Data
(63) Continuation of application No. PCT/US2024/012332, filed on Jan. 22, 2024.
(60) Provisional application No. 63/481,577, filed on Jan. 25, 2023.

(51) Int. Cl. A61F 2/24

(52) U.S. Cl. CPC A61F 2/2436

(57) ABSTRACT

A delivery system can comprise a delivery apparatus and a stabilizer assembly. The delivery apparatus can comprise a delivery shaft, a sleeve shaft, a pusher shaft, a hub assembly coupled to the pusher shaft, and a sleeve handle coupled to the sleeve shaft. The delivery shaft, sleeve shaft, and pusher shaft can be independently actuatable. The stabilizer assembly can include a hub assembly cradle configured to receive the hub assembly, a sleeve handle cradle configured to receive the sleeve handle, and a linear actuator configured to move the hub assembly cradle in an axial direction relative to the sleeve handle cradle. The stabilizer assembly can be configured to actuate the hub assembly relative to the sleeve handle while keeping the sleeve handle stationary, thereby stabilizing the delivery apparatus in such a way that allows for independent actuation of the delivery shaft, the sleeve shaft, and the pusher shaft.

### CROSS REFERENCE TO RELATED APPLICATION

This application is a continuation of PCT Patent Application No. PCT/US2024/012332 filed on Jan. 22, 2024, which application claims the benefit of U.S. Provisional Application No. 63/481,577, filed Jan. 25, 2023, each of these applications being incorporated by reference herein in its entirety.

### FIELD

The present disclosure relates to delivery systems for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (such as stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (such as through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

A docking device delivery system can be used to deliver a prosthetic medical device, such as a docking device used in conjunction with the prosthetic heart valve described above. The docking device can be positioned at the implantation site by the docking device delivery system to provide for better sealing between the implantation site and the prosthetic heart valve.

### SUMMARY

The foregoing and other objects, features, and advantages of the invention will become more apparent from the following detailed description, which proceeds with reference to the accompanying figures.

Described herein are prosthetic heart valves, delivery apparatuses, delivery systems, and methods for implanting prosthetic heart valves. The disclosed prosthetic heart valves, delivery apparatus, delivery systems, and methods can, for example, can provide for improved positioning of a docking device for use with a prosthetic heart valve. The docking device can be positioned using a docking device delivery apparatus comprising three independently actuatable shafts. The docking device delivery apparatus can be coupled to a stabilizer assembly that allows for improved positioning of the docking device by better stabilizing the docking device delivery apparatus during a docking device implantation procedure. As such, the devices and methods disclosed herein can, among other things, overcome one or more of the deficiencies of typical prosthetic heart valves and their delivery apparatuses and delivery systems.

A delivery system for a prosthetic medical device can comprise a handle and one or more shafts coupled to the handle.

In some examples, the delivery system can comprise three shafts.

In some examples, the one or more shafts can be independently actuated relative to one another.

In some examples, the one or more shafts can be coaxially aligned.

In some examples, the delivery system can comprise a stabilizer assembly for stabilizing the one or more shafts.

In some examples, the stabilizer assembly can comprise a hub assembly support.

In some examples, the stabilizer assembly can comprise a stabilizer track coupled to the hub assembly support.

In some examples, the hub assembly support can comprise a hub assembly cradle and a sleeve handle cradle.

In some examples, at least one of the hub assembly cradle and the sleeve handle cradle can be actuatable relative to the hub assembly support.

In some examples, the hub assembly support can comprise a brake configured to prevent relative movement between the hub assembly support to the stabilizer track.

In some examples, a delivery system can comprise a delivery shaft, a sleeve shaft disposed within the delivery shaft, and a pusher shaft disposed within the sleeve shaft. The delivery shaft, the sleeve shaft, and the pusher shaft can be independently actuated relative to one another. A proximal end portion of the delivery shaft can be coupled to a distal end portion of a handle configured to control an axial position of the delivery shaft. A proximal end portion of the sleeve shaft can be coupled to a distal end portion of a sleeve handle configured to control an axial position of the sleeve shaft. A proximal end portion of the pusher shaft can be coupled to a distal end portion of a hub assembly configured to control an axial position of the pusher shaft.

In some examples, a delivery system comprising a delivery shaft, a sleeve shaft disposed within the delivery shaft, and a pusher shaft disposed within the sleeve shaft can be configured to implant a docking device at a native heart valve. The delivery system can be configured to perform a variable encircling turn, during which radius of curvature of a distal end portion of the delivery system can be varied by actuating the pusher shaft in an axial direction relative to the delivery shaft and the sleeve shaft. Increasing the radius of curvature of the distal end portion of the delivery system can allow the delivery system to better encircle the chordae tendineae of the native heart valve, thereby better positioning the docking device between the implantation site and the prosthetic heart valve to further reduce the possibility of paravalvular leakage.

In some examples, a delivery system can comprise a delivery apparatus and a stabilizer assembly. The delivery apparatus can comprise a hub assembly and a sleeve handle and can be configured for use during a prosthetic medical device implantation procedure. The stabilizer assembly can comprise a hub assembly support configured to stabilize the hub assembly and the sleeve handle during the prosthetic medical device implantation procedure. The hub assembly support can comprise a hub assembly cradle, a sleeve handle cradle, and a linear actuator configured to move the hub assembly cradle in an axial direction relative to the sleeve handle cradle. When the hub assembly is disposed in the hub assembly cradle and the sleeve handle is disposed in the sleeve handle cradle, the hub assembly support can beneficially actuate the hub assembly relative to the sleeve handle while keeping the sleeve handle stationary, thereby further improving the stability of the delivery apparatus during the prosthetic medical device implantation procedure.

In some examples, a delivery system for delivering a prosthetic medical device comprises a delivery apparatus and a stabilizer assembly. The delivery apparatus can comprise a handle, a delivery shaft extending from a distal end portion of the handle and comprising a delivery shaft lumen extending along a length in the delivery shaft, a hub assembly extending from a proximal end portion of the handle, a sleeve shaft disposed within the delivery shaft lumen, wherein the sleeve shaft comprises a sleeve shaft lumen extending along the length of the sleeve shaft, a pusher shaft disposed within the sleeve shaft lumen, a sleeve handle coupled to a proximal end portion of the sleeve shaft, and a hub assembly coupled to a proximal end portion of the pusher shaft. The stabilizer assembly can be configured to stabilize the delivery apparatus and can comprise: a stabilizer track configured to be oriented in an axial direction and a hub assembly support configured to slidingly couple to the stabilizer track. The hub assembly support can comprise: a sleeve handle cradle configured to receive the sleeve handle, a hub assembly cradle configured to receive the hub assembly, wherein the hub assembly cradle is movable in the axial direction relative to the sleeve handle cradle, and a linear actuator coupled to the hub assembly, wherein the linear actuator is configured to actuate the hub assembly cradle in the axial direction relative to the sleeve handle cradle.

In some examples, a hub assembly support configured for use with a delivery system can comprise: a base portion; a housing disposed on the base portion and comprising an axially oriented slot, a sleeve handle cradle disposed on the housing and configured to receive a sleeve handle of the delivery system, a linear actuator coupled to the base portion, a traveler coupled to the linear actuator and extending through the axially oriented slot, and a hub assembly cradle coupled to the traveler, wherein the hub assembly cradle is configured to receive a hub assembly of the delivery system, and wherein the linear actuator is configured to actuate the hub assembly cradle in the axial direction relative to the sleeve handle cradle.

In some examples, a hub assembly support for use with a delivery system can comprise: a base portion and a linear actuator disposed on the base portion. The linear actuator can comprise: a threaded shaft oriented in an axial direction, a carriage operatively coupled to the threaded shaft, wherein the linear actuator is configured to actuate the carriage in the axial direction, a hub assembly cradle coupled to the carriage, wherein the hub assembly cradle is configured to receive a hub assembly of the delivery system, and a sleeve handle cradle disposed on the base portion, wherein the sleeve handle cradle is configured to receive a sleeve handle of the delivery system, wherein the linear actuator is configured to actuate the hub assembly cradle relative to the sleeve handle cradle in the axial direction.

In some examples, a delivery apparatus comprises one or more of the components recited in Examples 1-44 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

The above method(s) can be performed on a living animal or on a simulation, such as on a cadaver, cadaver heart, anthropomorphic ghost, simulator (for example, with body parts, heart, tissue, etc. being simulated).

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 schematically illustrates a stage in a docking device implantation procedure where a guide catheter is inserted into a blood vessel of a patient and navigated through the blood vessel and into a heart of the patient, according to one example.

FIG. 6 schematically illustrates another stage in the example docking device implantation procedure where a distal end portion of a docking device delivery apparatus is advanced from the guide catheter and into a left ventricle of the heart.

FIG. 7 schematically illustrates another stage in the example docking device implantation procedure where the distal end portion of the docking device delivery apparatus is coiled around a plurality of leaflets of the heart.

FIG. 8 schematically illustrates another stage in the example docking device implantation procedure where a radius of curvature of the distal end portion of the docking device delivery apparatus is increased to encircle the chordae tendineae of the heart in a variable encircling turn.

FIG. 9 schematically illustrates another stage in the example docking device implantation procedure where a sleeve shaft of the docking device delivery apparatus is retracted in a proximal direction to unsheathe a guard member of a docking device.

FIG. 10 schematically illustrates another stage in the example docking device implantation procedure where the sleeve shaft is advanced in the distal direction to foreshorten the guard member.

FIG. 11 schematically illustrates another stage in the example mitral valve replacement procedure where the docking device delivery apparatus is decoupled from the docking device.

FIG. 12 is a perspective view of a docking device delivery system configured for use during the docking device implantation procedure of FIGS. 5-11, according to one example.

FIG. 13 is a top view of a docking device delivery apparatus for use with the docking device delivery system of FIG. 12, according to one example.

FIGS. 14A-14B are perspective views of a hub assembly support for use with the docking device delivery system of FIG. 12, according to one example.

FIG. 15 is an internal view of the hub assembly support, according to one example.

FIGS. 16A-16E are side views of the docking device delivery system of FIG. 12 during the example mitral valve replacement procedure of FIGS. 5-11.

FIG. 17 is a perspective view of the hub assembly support, according to a second example.

FIG. 18 is a perspective view of the hub assembly support, according to a third example.

FIG. 19 is a side view of a guide catheter for use with the docking device delivery system of FIG. 12, according to one example.

FIG. 20 is a side view of the guide catheter and the docking device delivery apparatus, according to one example.

FIG. 21 is a side view of the guide catheter and a docking device delivery apparatus, according to a second example.

FIG. 22 is a perspective view of a docking device for use with the docking device delivery system of FIG. 12, according to one example.

FIG. 23 is a perspective view of a prosthetic heart valve delivery apparatus, according to one example.

FIG. 24 is a perspective view of a prosthetic heart valve configured for use with the prosthetic heart valve delivery apparatus of FIG. 23, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (such as out of the patient's body), while distal motion of the device is motion of the device away from the user and toward the implantation site (such as into the patient's body). The terms “longitudinal” and “axial” refer to an axis extending in the proximal and distal directions, unless otherwise expressly defined.

The terms “lateral” and “radial” refer to an axis perpendicular to the longitudinal axis. When referring to a “lateral” direction with respect to a stabilizer assembly for a docking device delivery system, the term “lateral” refers to an axis that perpendicular to the longitudinal axis and parallel with a plane defined by a stabilizer track of the stabilizer assembly.

As used herein, “e.g.” means “for example,” and “i.e.” means “that is.”

### Introduction to the Disclosed Technology

Disclosed herein are examples of a delivery system that can be used to navigate a subject's vasculature to deliver a prosthetic medical device (such as a docking device used in conjunction with a prosthetic heart valve), tools, agents, or other therapy to a target implantation site within the body of the subject.

In connection therewith, various systems are described herein that, in some examples, can stabilize and actuate various components of the delivery system to better improve the positioning of the prosthetic.

The delivery system can comprise a plurality of shafts that can be independently actuated relative to one another. In some examples, the delivery system can comprise a delivery shaft comprising a delivery shaft lumen, a sleeve shaft disposed within the delivery shaft lumen and comprising a sleeve shaft lumen, and a pusher shaft disposed within the sleeve shaft lumen. The prosthetic can be positioned at the target implantation site by actuating the pusher shaft relative to the delivery shaft and the sleeve shaft.

Disclosed herein are exemplary devices and/or methods that can, among other things, make it easier to actuate (for example, axially move) one or more components of a delivery system relative to one or more other components of the delivery system.

### Examples of the Disclosed Technology

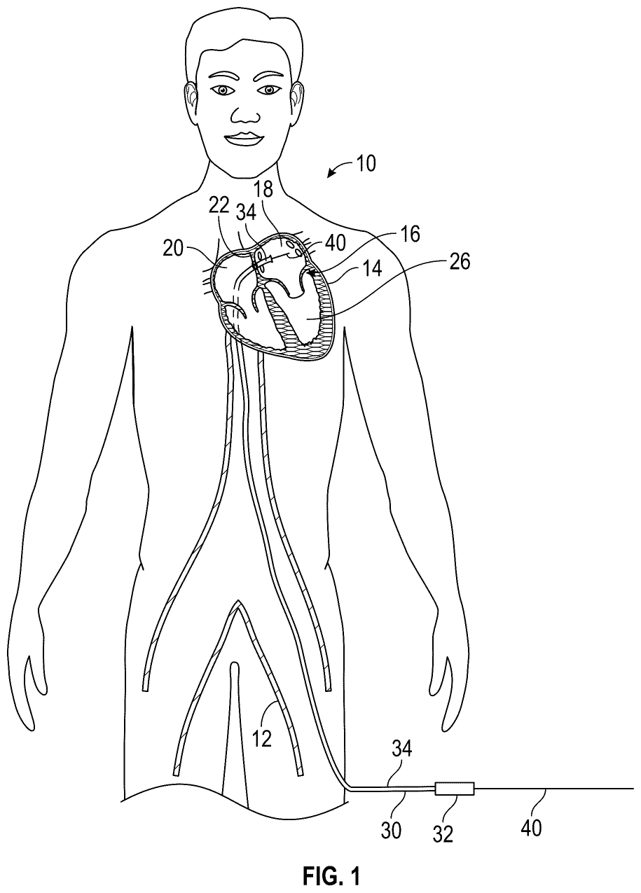

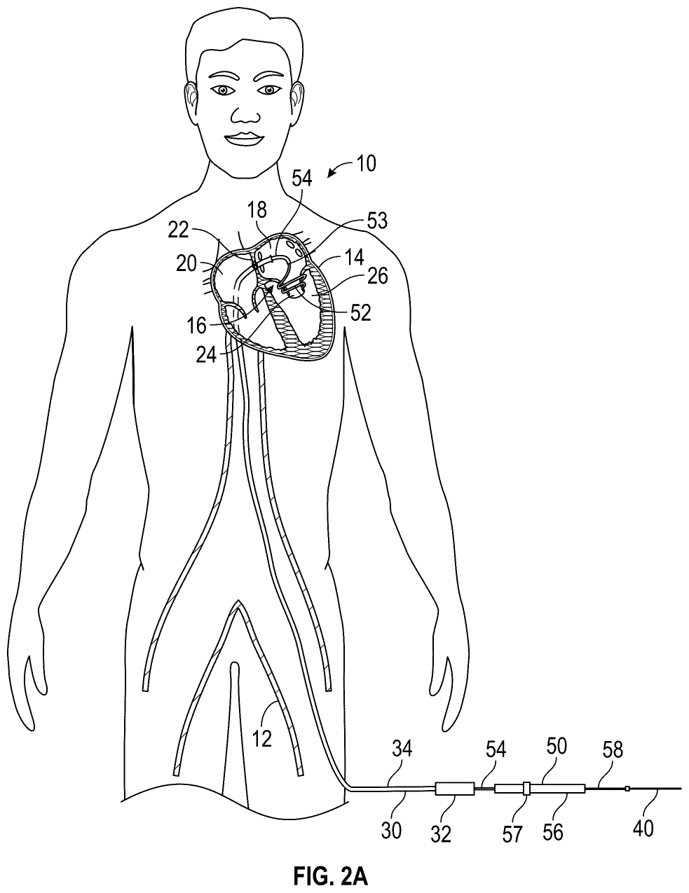

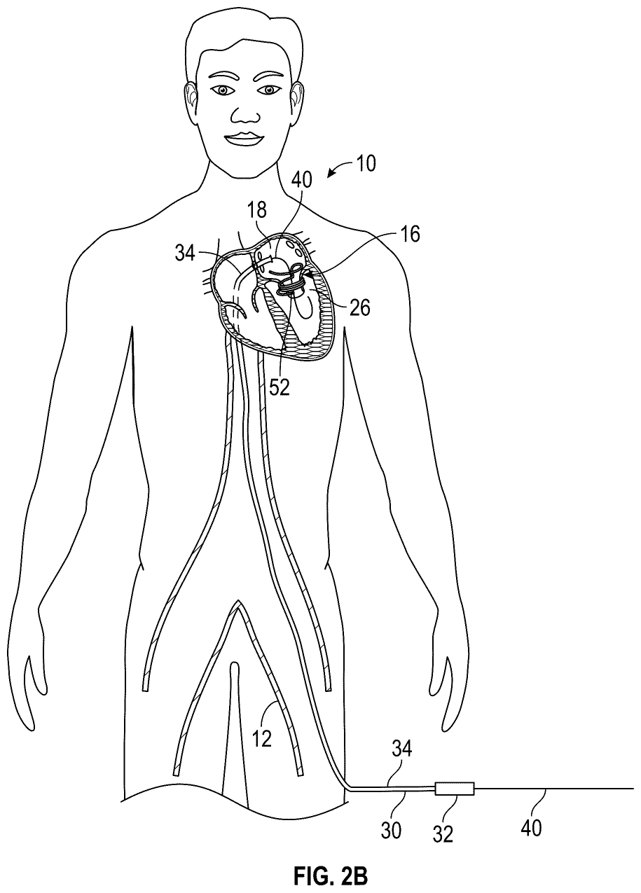

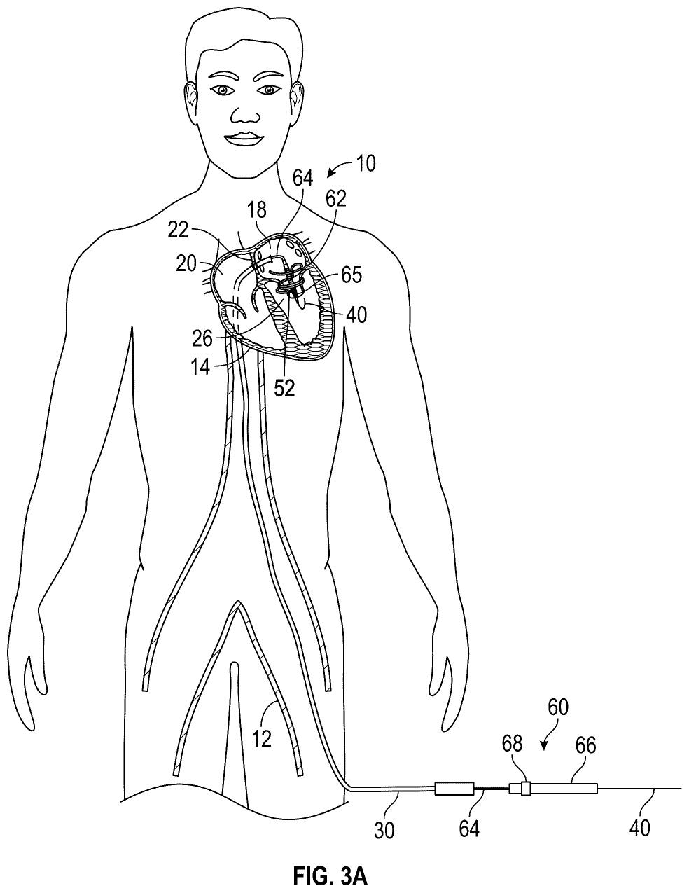

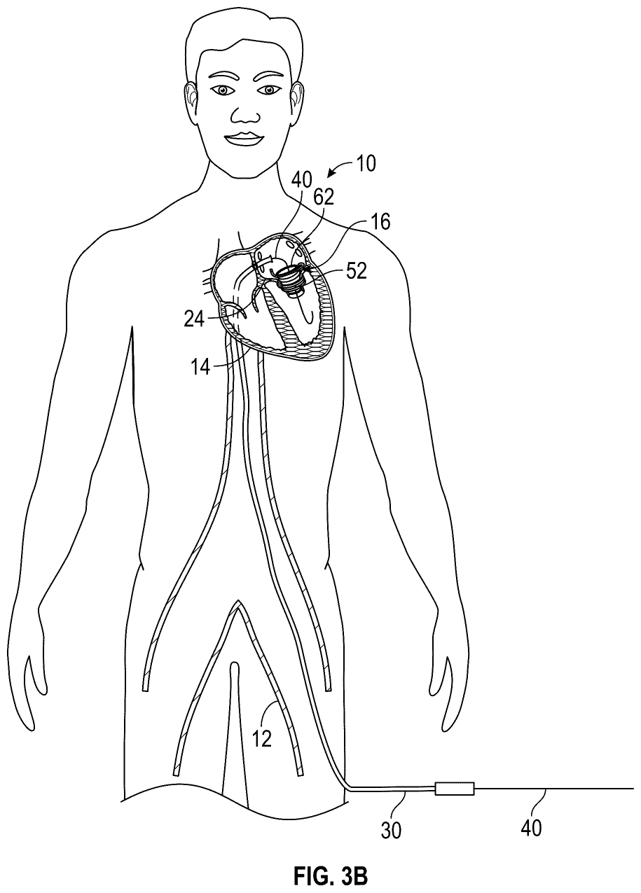

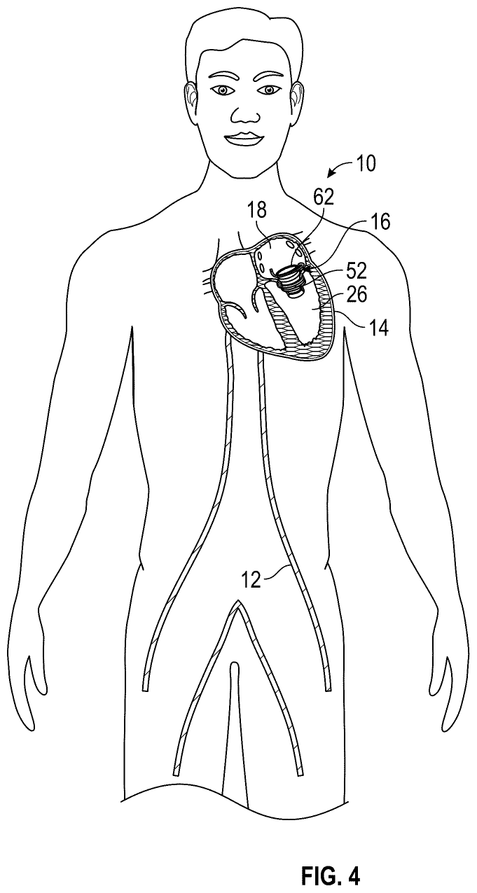

FIGS. 1-4 depict an example of a transcatheter heart valve replacement procedure (such as a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a delivery apparatus 50 (FIG. 2A) and then removes the delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (such as the delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 (which may also be referred to as a catheter shaft 34) extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (such as the guide catheter 30, the delivery apparatus 50, the prosthetic valve delivery apparatus 60, additional catheters, or the like) and their associated devices (such as docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle 26 of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (such as through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a delivery apparatus 50 (which may also be referred to as an “implant catheter,” a dock delivery system,” a “docking device delivery apparatus,” and/or a “docking device delivery device”).

In general, the delivery apparatus 50 comprises a delivery shaft 54 (which may also be referred to as a “dock delivery system shaft”), a handle 56 (which may also be referred to as a “dock delivery system handle”), and a pusher assembly 58. The delivery shaft 54 is configured to be advanced through the patient's vasculature (blood vessel 12) and to the implantation site (such as native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the delivery apparatus 50 is configured to be gripped and/or otherwise held by the user, outside the body of the patient 10, to advance the delivery shaft 54 through the patient's vasculature (such as the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (such as the native mitral valve 16). For example, the pusher assembly 58 is configured to be adjusted by the user to push the docking device 52 out of the distal end portion 53 of the delivery shaft 54. A shaft (which may also be referred to as a “pusher shaft”) of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the delivery apparatus 50 such that the docking device 52 can be released after being deployed at the native mitral valve 16.

Further details of the docking device delivery apparatus and its variants are described in International Publication No. WO2020/247907 and U.S. Provisional Patent Application Nos. 63/363,162 and 63/380,796, which are incorporated by reference herein in their entirety.

Referring again to FIG. 2A, after the guide catheter 30 is positioned within the left atrium 18, the user may insert the delivery apparatus 50 (such as the delivery shaft 54) into the patient 10 by advancing the delivery shaft 54 of the delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the delivery apparatus 50 through the blood vessel 12 along the guidewire 40 until the delivery shaft 54 reaches the left atrium 18, as illustrated in FIG. 2A. Specifically, the user may advance the delivery shaft 54 of the delivery apparatus 50 by gripping and exerting a force on (for example, by pushing) the handle 56 of the delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (such as the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (such as the portion of the docking device 52 shown in FIG. 2A that is configured to be positioned within the left ventricle 26 and/or on the ventricular side of the native mitral valve 16), the user may then deploy the remaining portion of the docking device 52 (such as an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the delivery apparatus 50 from the docking device 52. Once the docking device 52 is disconnected from the delivery apparatus 50, the user may retract the delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the delivery apparatus 50 (including the delivery shaft 54) has been removed from the patient 10 such that only the guidewire 40 and the guide catheter 30 remain inside the patient 10. In some examples, after removing the delivery apparatus 50, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (such as the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, by pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, by inflating the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (such as tricuspid, pulmonary, and/or aortic valves). Further, the same and/or similar delivery apparatuses (such as the delivery apparatus 50, prosthetic valve delivery apparatus 60, guide catheter 30, and/or guidewire 40), docking devices (such as the docking device 52), replacement heart valves (such as the prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

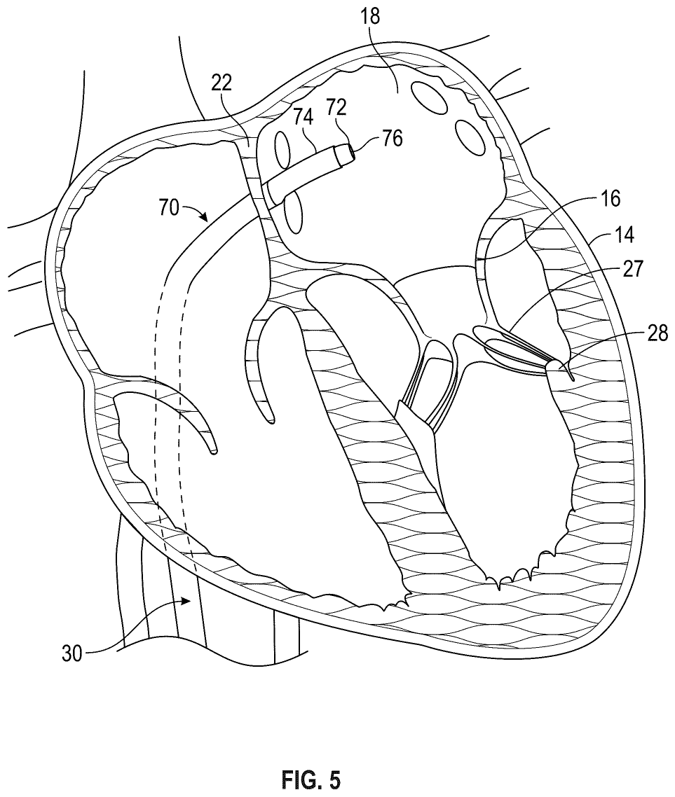

FIGS. 5-11 schematically illustrate a procedure for implanting a prosthetic medical device at a target implantation site in a subject (such as the patient 10). In some examples, the procedure is a docking device implantation procedure for implanting a docking device 152 at an annulus of the native mitral valve 16 of the patient 10. In some examples, one exemplary difference between the docking device 52 of FIGS. 1-4 and the docking device 152 of FIGS. 5-11 can be that the docking device 152 optionally includes a guard member 180 coupled to the docking device 152, wherein the guard member 180 can be configured to further mitigate the possibility of paravalvular leakage between the annulus of the native mitral valve 16 and a prosthetic heart valve (such as the prosthetic heart valve 62) positioned in the docking device 152.

The procedure of FIGS. 5-11 can be performed using a delivery apparatus 150 (which may also referred to as a “docking device delivery apparatus”). In some examples, one exemplary difference between the delivery apparatus 150 and the delivery apparatus 50 of FIGS. 1-4 is that the delivery apparatus 150 can comprise three independently actuatable shafts: a delivery shaft 154 (which may also be referred to as a “dock delivery system shaft”), a sleeve shaft 182, and a pusher shaft 184 (which may also be referred to as a “dock shaft”). The pusher shaft 184 can be disposed within the sleeve shaft 182, which can in turn be disposed within the delivery shaft 154. In some examples, the delivery shaft 154, the sleeve shaft 182, and the pusher shaft 184 can be coaxial. The delivery shaft 154, the sleeve shaft 182, and the pusher shaft 184 can be independently actuated relative to one another in the axial direction during the docking device implantation procedure to better position the docking device 152 within the annulus of native mitral valve 16, such that the implanted docking device 152 can better encircle one or more chordae tendineae 27 of the heart 14 and provide for better sealing between the implantation site and a prosthetic heart valve (such as the prosthetic heart valve 62).

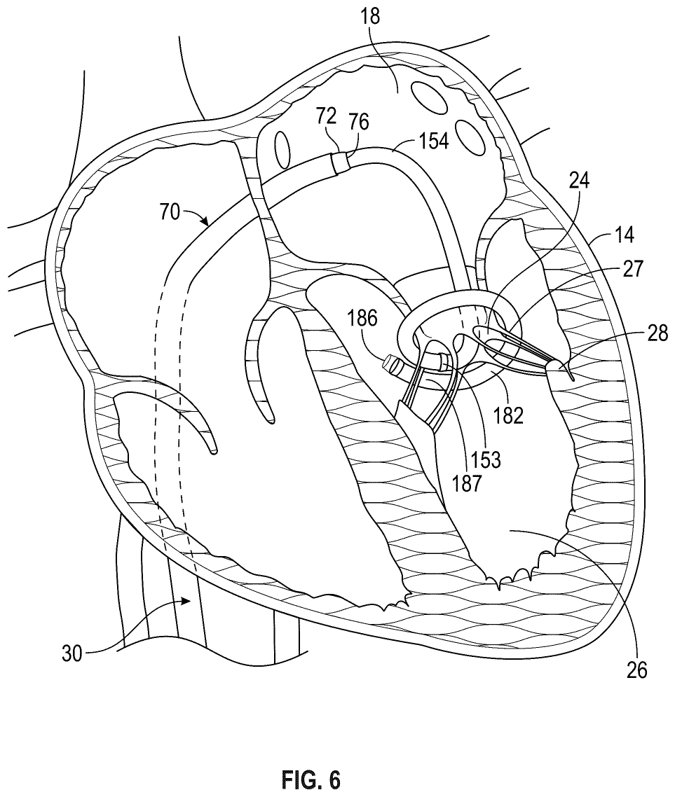

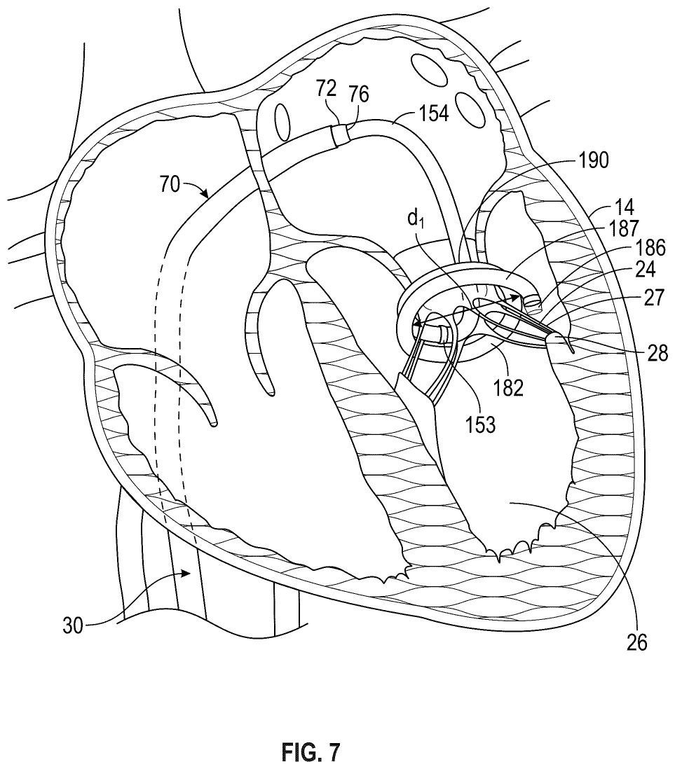

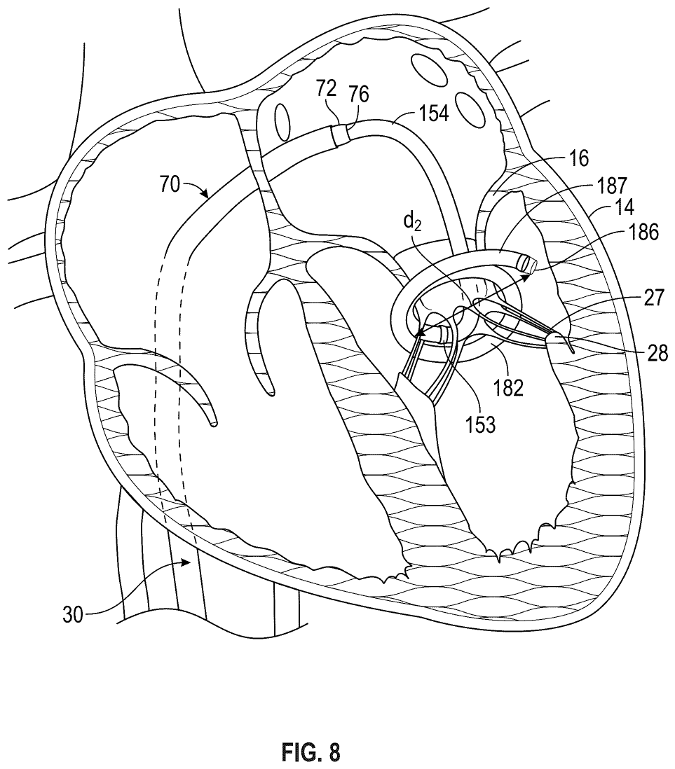

During the procedure, a user of the delivery apparatus 150 first creates a pathway to a patient's native heart valve using the guide catheter 30 (FIG. 5). The user then distally advances a distal end portion of the delivery apparatus 150 to advance the docking device 152 to the target implantation site (FIG. 6-7). In some examples of the procedure, the user can acuate the delivery system to change or adjust the curvature of the distal end portion of the delivery apparatus 150 (see, for example, the leading turn 187 of the delivery apparatus 150 in FIGS. 7-8). This adjustable radius of curvature can be referred to as a “variable encircling turn” (VET). The VET can, for example, make it easier to encircle one or more chordae tendineae 27 connecting the leaflets 24 to the papillary muscles 28 of the heart 14 by proximally retracting the pusher shaft 184 relative to the sleeve shaft 182.

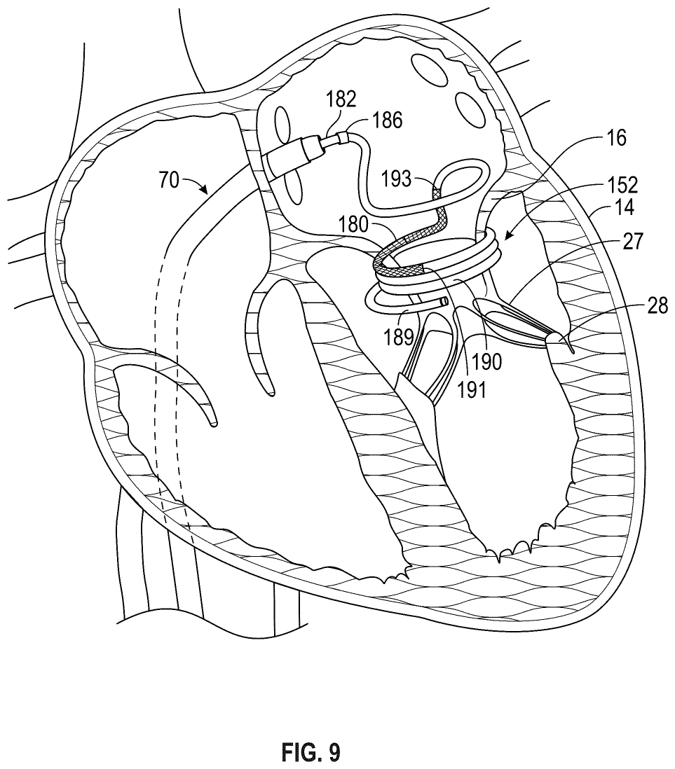

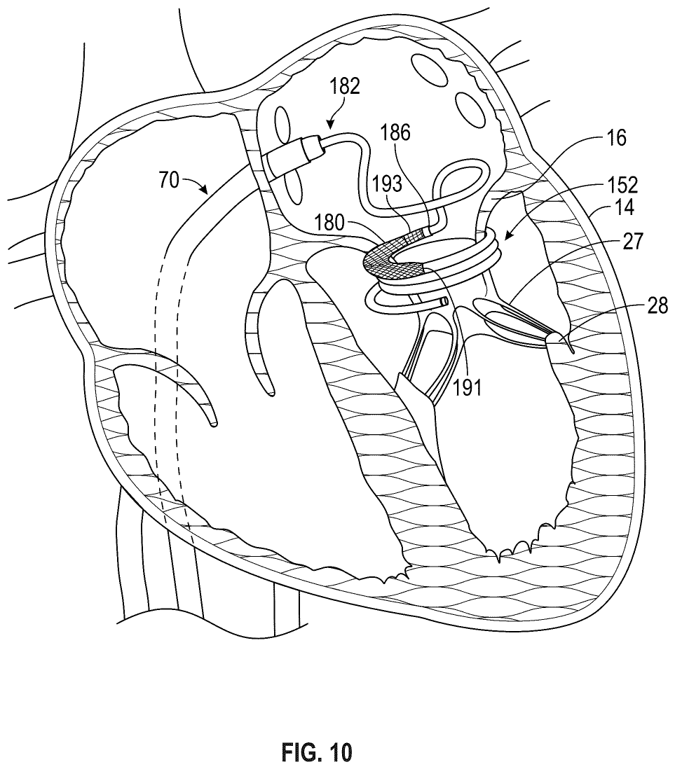

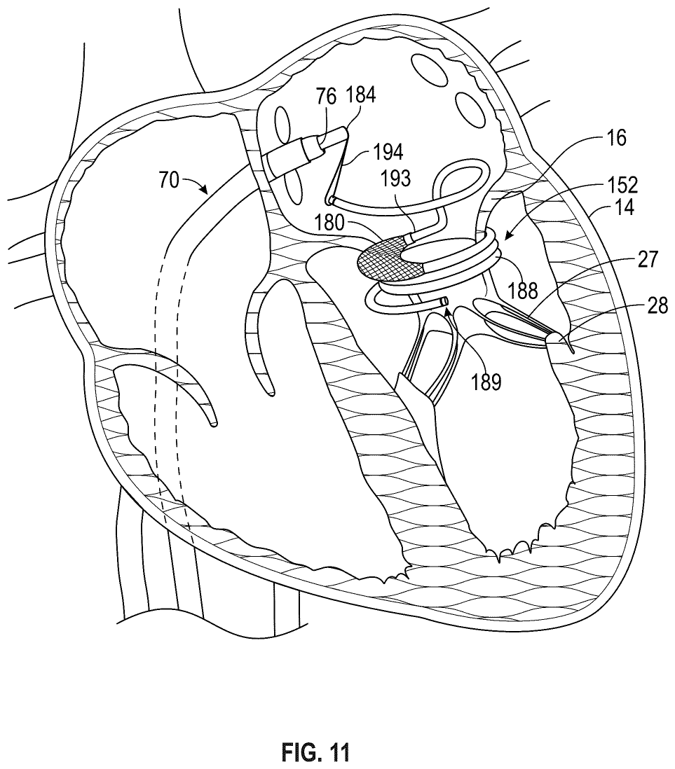

In some examples where the docking device 152 further comprises a guard member 180, the user can then retract the delivery shaft 154 and the sleeve shaft 182 in the proximal direction to expose the guard member 180 (FIG. 9) from the sleeve shaft 182. In some examples, the user can then advance the sleeve shaft 182 in the distal direction to exert an axially compressive force against the guard member 180, thereby axially foreshortening and radially expanding the guard member 180 (FIG. 10). Finally, the user can decouple the docking device 152 from the pusher shaft 184 and remove the delivery device system 150 from the patient 10 (FIG. 11).

FIG. 5 illustrates a stage in the procedure in which the guide catheter 30 is advanced in a distal direction through the patient's vasculature and into the left atrium 18 of the heart 14. The guide catheter 30 comprises the catheter shaft 34 that includes a distal end 72, a flex region 74, and a lumen exit 76 on the distal end 72 of the catheter shaft 34. The lumen exit 76 is connected to a catheter shaft lumen disposed within the catheter shaft 34. A delivery apparatus (such as any of the prosthetic device delivery apparatuses or implant catheters described herein) is configured to be disposed within the catheter shaft lumen. The catheter shaft lumen extends from a proximal end portion of the catheter shaft 34 (such as the portion of the catheter shaft 34 coupled to the handle 32) to the lumen exit 76. The guide catheter 30 is positioned such that the distal end 72 of the catheter shaft 34 is disposed within the left atrium 18 of the heart 14.

In some examples, the catheter shaft 34 can comprise one or more pull wires for adjusting a curvature of the flex region 74 of the catheter shaft 34. In some examples, the pull wires can extend through a lumen coupled to the lumen exit 76 and can couple to a portion of the catheter shaft 34, such as a pull wire ring at or adjacent the distal end 72. In some examples, the pull wires can extend through one or more pull wire lumens embedded in the catheter shaft 34. In some examples, adjusting a tension of the pull wires can adjust the curvature of a flex region 74 of the catheter shaft 34. In some instances, the catheter shaft 34 (including its flex region 74) can be integrally formed as a single, unitary component. In some instances, the catheter shaft 34 can comprise one or more segments (for example, the flex region 74, other regions, etc.) that are formed as separate components that are coupled together (such as via fasteners, adhesive, mating features, and/or other means for coupling). In some examples, the flex region 74 can comprise a material that is more prone to flexing, bending, twisting, etc. than the remaining portion of the catheter shaft 34 (for example, a polymer having relatively lower durometer hardness). This can enable the curvature of the flex region 74 to be adjusted or increased at a different rate than the remaining portion of the catheter shaft 34 when the pull wires are tensioned. For example, the curvature of the flex region 74 can change at an increased rate relative to the proximal portion of the catheter shaft 34 as the tension of the pull wires is increased. The catheter shaft 34 can also include one or more reinforcing braids or jackets that makes the catheter shaft 34 more resistant to flexing, bending, twisting, etc., for example, to prevent one or more of the lumens from kinking or collapsing when the catheter shaft 34 is manipulated.

During this stage, the docking device 152 is disposed within the sleeve shaft 182, which in turn is disposed within the delivery shaft 154, which in turn is disposed within the catheter shaft 34. The pusher shaft 184 is disposed proximally adjacent the docking device 152 within the sleeve shaft 182. In some examples, the docking device 152, the sleeve shaft 172, the delivery shaft 154, and the catheter shaft 34 can be coaxially aligned. During this stage, the docking device 152 is in a generally straight delivery configuration (i.e., without any coiled or looped portions, but can be flexed or bent) so as to maintain a small radial profile when moving through a patient's vasculature.

FIG. 6 illustrates a stage in the procedure in which the docking device 152, the delivery shaft 154, the sleeve shaft 182, and the pusher shaft 184 are advanced in a distal direction through the lumen exit 76 of the catheter shaft 34, through the left atrium 18, and to the native mitral valve 16. The docking device 152 is disposed within a sleeve shaft lumen of the sleeve shaft 182, which in turn is disposed within a delivery shaft lumen of the delivery shaft 154. The pusher shaft 184 is disposed proximally adjacent the docking device 152 within the sleeve shaft 182.

The delivery shaft 154, which in some examples can resemble the delivery shaft 54, comprises the delivery shaft lumen through which the sleeve shaft 182 and the pusher shaft 184 can extend. The delivery shaft lumen is configured to extend in the axial direction along the length of the delivery shaft 154 between a handle of the delivery apparatus 150 and a distal end portion 153 of the delivery shaft 154. The sleeve shaft 182 and the pusher shaft 184 are configured to exit the delivery shaft lumen through an opening at the distal end portion 153.

The sleeve shaft 182 is configured to extend through the delivery shaft 154 and sheathe the docking device 152 and at least a portion of the pusher shaft 184 as the docking device 152 is navigated through the patient's vasculature to the native mitral valve 16. The sleeve shaft 182 comprises the sleeve shaft lumen extending along the length of the sleeve shaft 182 between a handle of the delivery apparatus 150 and a distal end portion 186 of the sleeve shaft 182. In some examples, a portion (for example, a proximal end portion) of the sleeve shaft 182 can have a substantially U-shaped axial cross-section or other shape that allows the proximal end portion of the pusher shaft 184 to exit the sleeve shaft 182. The distal end portion of the pusher shaft 184 can exit the sleeve shaft 182 at an opening at the distal end portion 186 of the sleeve shaft 182.

The distal end portion 186 of the sleeve shaft 182 is configured to capture the native tissue (for example, the native leaflets 24 and chordae 27). The sleeve shaft 182 can have a relatively low-friction and/or lubricious outer surface to reduce the likelihood of the sleeve shaft 182 snagging on the native tissue.

In some examples, the sleeve shaft 182 can comprise a plurality of layers. For example, the sleeve shaft 182 can comprise an inner-most polymeric layer, a braided or other type of flexible reinforcing layer, and an outer-most polymeric layer. In some examples, the reinforcing layer is a shape memory material and/or elastic material (for example, nitinol and/or stainless steel).

In some instances, the distal end portion 186 of the sleeve shaft 182 can be curved to help facilitate the capture of the native tissue. This can be accomplished by forming the distal end portion 186 of the sleeve shaft 182 in a curved configuration and/or by forming the sleeve shaft 182 of a relatively more flexible material than the docking device 152 and advancing the curved docking device 152 into the sleeve shaft 182, which can result in the sleeve shaft 182 assuming a curved configuration and/or the curvature of the sleeve shaft 182 being altered by the docking device 152.

In this manner, the distal end portion 186 of the sleeve shaft 182 can form a sleeve shaft leading turn 187 configured to capture the chordae tendineae 27 as the sleeve shaft 182 is advanced around the leaflets 24 of the native mitral valve 16. The sleeve shaft leading turn 187 is a portion of the sleeve shaft 182 disposed at or adjacent the distal end portion 186 that comprises a curved portion of the sleeve shaft 182 having a radius of curvature. When the docking device 152 is not sheathed within the portion of the sleeve shaft 182 corresponding to the sleeve shaft leading turn 187, the sleeve shaft leading turn 187 has a radius of curvature equal to a first radius of curvature (r₁) As discussed later in this application, particularly in reference to FIGS. 7-8, the radius of curvature of the sleeve shaft leading turn 187 can be varied by relative movement between the sleeve shaft 182 and the docking device 152. In some examples, in which the sleeve shaft 182 can be constructed from, formed of, and/or comprise a shape memory material, the sleeve shaft 182 may originally be formed such that the sleeve shaft leading turn 187 has the first radius of curvature (r₁). The sleeve shaft leading turn 187 can be forced into another configuration having another radius of curvature (for example, second radius of curvature (r₂)) but can revert to its original configuration having the first radius of curvature (r₁) when the force is removed. In some examples, the second radius of curvature (r₂) can be less than the first radius of curvature (r₁).

In some examples, the sleeve shaft leading turn 187 can conform to a shape or curvature of another component (such as the docking device 152) sheathed by the sleeve shaft leading turn 187, such that the radius of curvature of the sleeve shaft leading turn 187 is equal to a corresponding radius of curvature of the other component. As such, the distal end portion 186 of the sleeve shaft 182 can have a lesser radius of curvature when a distal end portion (such as leading turn 189) of the docking device 152 is disposed at or proximate to the distal end portion 186 of the sleeve shaft 182. This is because the docking device 152 can have a smaller radius of curvature and can be relatively more rigid than the sleeve shaft 182. In some examples, the radius of curvature of the distal end portion 186 of the sleeve shaft 182 can be increased by moving the distal end of the docking device 152 proximally relative to the distal end portion 186 of the sleeve shaft 182 such that the sleeve shaft 182 can assume its pre-set configuration. This can be done by moving the docking device 152 proximally while maintaining the position of the sleeve shaft 182, by moving the sleeve shaft 182 distally relative to the docking device 152, or a combination of the two.

The pusher shaft 184 is configured to extend through the delivery shaft 154 and the sleeve shaft 182. The pusher shaft 184 is configured to be disposed proximally adjacent the docking device 152 within the sleeve shaft 182 while the docking device 152 is navigated through the patient's vasculature to the native mitral valve 16. When the pusher shaft 184 is moved in the axial direction relative to the sleeve shaft 182, the pusher shaft 184 can exert a force on the docking device 152 to move the docking device 152 in the axial direction. In some examples, the docking device 152 can be releasably coupled to the pusher shaft 184 via a connection mechanism of the delivery apparatus 150 such that the docking device 152 can be released after being deployed at the native mitral valve 16.

In some examples, the distal end portion 153 of the delivery shaft 154 can be positioned during this stage between the leaflets 24 of the native mitral valve 16 (such as at or near the posteromedial commissure). In some examples, the distal end portion 153 of the delivery shaft 154 can extend distally past the native mitral valve 16 and be positioned adjacent the native mitral valve 16 in the left ventricle 26. In some examples, the distal end portion 153 of the delivery shaft 154 can be positioned adjacent the native mitral valve 16 in the left atrium 18.

Once the distal end portion 153 of the delivery shaft 154 is positioned, the docking device 152, the sleeve shaft 182, and the pusher shaft 184 are advanced in a distal direction out of the opening at the distal end portion 153 of the delivery shaft 154, through the native mitral valve 16, and into the left ventricle 26.

FIG. 7 illustrates a stage in the procedure in which the docking device 152 (disposed within the sleeve shaft 182), the sleeve shaft 182, and the pusher shaft 184 (disposed within the sleeve shaft 182 and proximally adjacent the docking device 152) wrap around or encircle the leaflets 24 on the ventricular side of the native mitral valve 16. When the docking device 152 exits the delivery shaft 154, the docking device 152 assumes a coiled configuration that is configured to wrap around or encircle the leaflets 24 on the ventricular side of the native mitral valve 16. In some examples, in which the docking device 152 can be constructed from, formed of, and/or comprise a shape memory material, the docking device 152 may originally be formed in the coiled configuration, but may be forced into a straightened delivery configuration by the delivery shaft 154. The docking device 152 can assume its original coiled configuration once the docking device 152 is no longer sheathed by the delivery shaft 154.

As previously discussed, the portions of the sleeve shaft 182 that sheathe the docking device 152 can conform to or assume the shape and/or curvature of corresponding portions of the docking device 152. For example, the sleeve shaft leading turn 187 can conform to the leading turn 189 of the docking device 152, wherein the leading turn 189 has radius of curvature equal to the second radius of curvature (r₂). Thus, the sleeve shaft leading turn 187 can assume a configuration having the second radius of curvature (r₂). In other words, the variable encircling turn can be equal to the second radius of curvature (r₂).

FIG. 8 illustrates an optional stage in the procedure in which the radius of curvature of the sleeve shaft leading turn 187 (in other words, the variable encircling turn) is increased from the second radius of curvature (r₂) to the first radius of curvature (r₁) to better capture the chordae tendineae 27 within the docking device leading turn 189. In some examples, the radius of curvature of the sleeve shaft leading turn 187 can be increased by retracting the pusher shaft 184 in the proximal direction relative to the sleeve shaft 182, such that docking device leading turn 189 and/or the docking device 152 is no longer sheathed by the sleeve shaft leading turn 187. In some examples, the radius of curvature of the sleeve shaft leading turn 187 can be increased by advancing the distal end portion 186 of the sleeve shaft 182 in the distal direction relative to the docking device 152. When the sleeve shaft leading turn 187 is no longer forced to conform to the curvature of the docking device leading turn 189 having the second radius of curvature (r₂), the sleeve shaft leading turn 187 can revert to its original configuration having the first radius of curvature (r₁), which is larger than the second radius of curvature (r₂). Since the chordae tendineae 27 are captured within the sleeve shaft leading turn 187, increasing the variable encircling turn to the larger first radius of curvature (r₁) beneficially allows for more portions of the chordae tendineae 27 to be captured by the sleeve shaft leading turn 187 as it is advanced around the leaflets 24.

During the step illustrated in FIG. 8, the delivery shaft 154 can be kept stationary to preserve the position of the distal end portion 153 of the delivery shaft 154 relative to the native mitral valve 16 (such as at or near the posteromedial commissure). In some examples, the sleeve shaft 182 can be kept stationary to preserve the encircling position and/or radial orientation of the sleeve shaft 182 relative to the native mitral valve 16. In some examples, the docking device 152 and/or the pusher shaft 184 can be kept stationary while the sleeve shaft 182 is moved during this step. In some examples, neither the sleeve shaft nor the pusher shaft 184 are kept stationary during this step.

As shown in FIG. 8, the variable encircling turn can be adjusted after the sleeve shaft 182 has made one helical turn around the leaflets 24. However, in some examples, the variable encircling turn can be adjusted after the sleeve shaft 182 has formed a plurality of helical turns around the leaflets 24. In some examples, the variable encircling turn can be adjusted before any helical turns have been formed around the leaflets 24.

FIG. 9 illustrates an optional stage in the procedure in which the delivery shaft 154 and the sleeve shaft 182 are retracted in the proximal direction to unsheathe the guard member 180. The docking device 152 comprises a coil 188 that defines a central region 190 comprising a plurality of helical turns wrapped around the leaflets 24 and a docking device leading turn 189 extending from a distal end portion of the central region 190.

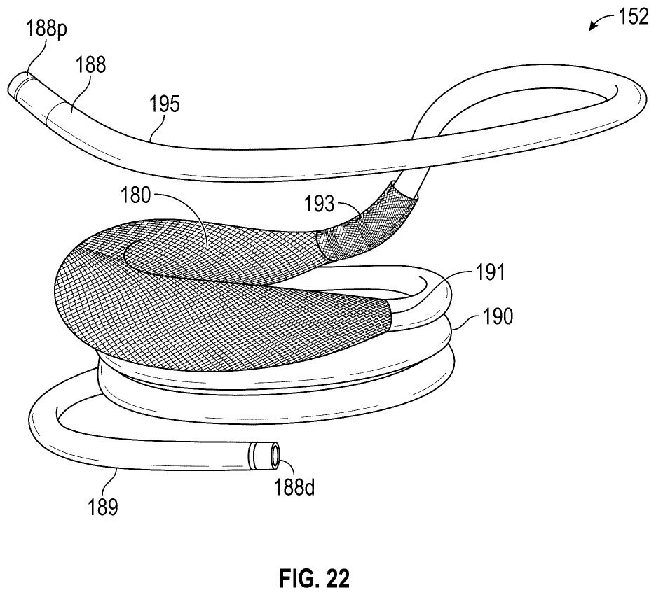

The docking device 152 can further comprise the guard member 180 disposed on the docking device 152 such that the guard member 180 is positioned at or near the native mitral valve 16 (such as at or near the posteromedial commissure) when the docking device 152 is implanted at the native mitral valve 16. In some examples, the guard member 180 can be disposed proximally adjacent a central region (FIG. 22), wherein the central region can comprise a plurality of helical turns when the docking device 152 is wrapped around the leaflets 24. The guard member 180 can extend between a distal end portion 191 that is fixedly coupled to the docking device 152 and a movable proximal end portion 193 that can be moved along at least a portion of the docking device 152 in the axial direction. In some examples, the distal end portion 191 of the guard member 180 can abut the central region 190.

During the stages of the docking device implantation procedure shown in FIGS. 5-8, the guard member 180 can be covered by the delivery shaft 154 and the sleeve shaft 182. However, during the stage illustrated in FIG. 9, relative movement between the delivery shaft 154, the sleeve shaft 182, and the pusher shaft 184 can unsheathe the guard member 180. In some examples, the sleeve shaft 182 can be retracted in the proximal direction from the left ventricle 26, through the mitral valve 16, and into the left atrium 18 such that the distal end portion 186 of the sleeve shaft 182 is proximally closer to the user than the proximal end portion 193 of the guard member 180. In some examples, the distal end portion 186 of the sleeve shaft 182 can be distally disposed relative to the lumen exit 76. In some examples, the guard member 180 can be unsheathed by advancing the pusher shaft 184 distally relative to the sleeve shaft 182.

In some examples, the delivery shaft 154 can be retracted through the left atrium 18 in the proximal direction such that the distal end portion 153 of the delivery shaft 154 is proximally closer to the user than the proximal end portion 193 of the guard member 180. In some examples, the delivery shaft 154 can be retracted through the lumen exit 76 and into the catheter shaft lumen of the catheter shaft 34. In some examples, the pusher shaft 184 can be advanced distally relative to the delivery shaft 154 such that the distal end portion 153 of the delivery shaft 154 is proximally disposed relative to the guard member 180.

FIG. 10 illustrates an optional “seating” stage in the procedure in which the sleeve shaft 182 is distally advanced relative to the docking device 152 to axially foreshorten and radially expand the guard member 180. In some examples, the sleeve shaft 182 can be advanced in the distal direction such that the distal end portion 186 of the sleeve shaft 182 abuts and contacts the proximal end portion 193 of the guard member 180. In some examples, the pusher shaft 184 (and the docking device 152 coupled to the pusher shaft 184) can be retracted in the proximal direction such that the distal end portion 186 of the sleeve shaft 182 abuts and contacts the proximal end portion 193 of the guard member 180. The sleeve shaft 182 exerts a force upon the guard member 180 to distally advance the proximal end portion 193 of the guard member 180 relative to the docking device 152. Since the distal end portion 191 of the guard member 180 is fixedly coupled to the docking device 152, exerting the force upon the guard member 180 axially foreshortens and radially expands the guard member 180 to a deployed configuration. When in the deployed configuration, the guard member 180 further reduces the possibility of paravalvular leakage between the native mitral valve 16 and a prosthetic heart valve (such as the prosthetic heart valve 62). The frictional engagement between the proximal end of the guard member 180 and the docking device 152 can retain the position of the guard member 180 relative to the docking device 152 when the sleeve shaft 182 is retracted from the proximal end of the guard member 180.

FIG. 11 illustrates a stage in the procedure in which the delivery apparatus 150, including the delivery shaft 154 and the sleeve shaft 182, are retracted through the catheter shaft lumen of the catheter shaft 34. In some examples, the docking device 152 can be connected to the pusher shaft 184 via a release suture 194 that can be configured to be tied to the docking device 152. The release suture 194 can be cut during this stage to release the docking device 152 from the delivery apparatus 150.

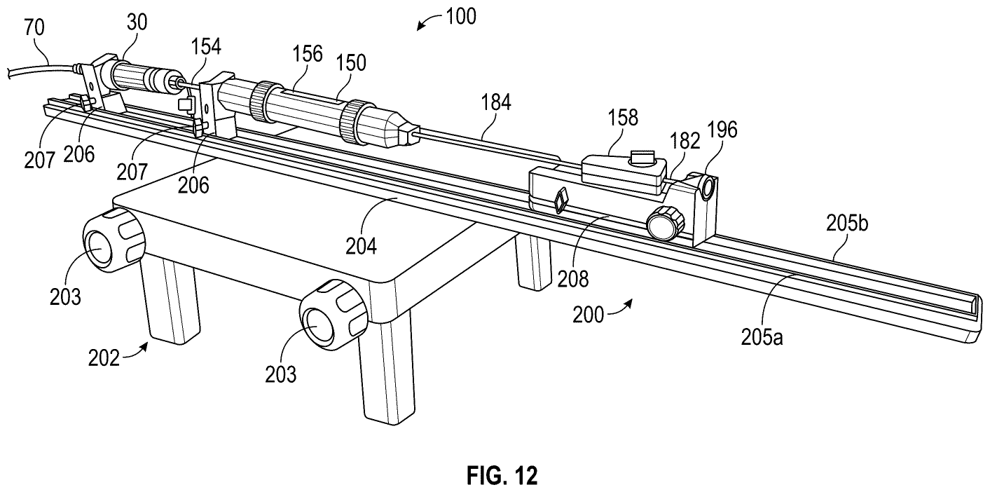

FIG. 12 illustrates an example delivery system 100 (which can also be referred to as a “docking device delivery system”) that can be used in the procedure for implanting a prosthetic medical device, as described above with reference to FIGS. 5-11. The delivery system 100 includes the delivery apparatus 150, the guide catheter 30, and a stabilizer assembly 200 (which may also be referred to as a “stabilizing tower” or “stabilizing device”) configured to stabilize the delivery apparatus 150 and/or the guide catheter 30 during the procedure.

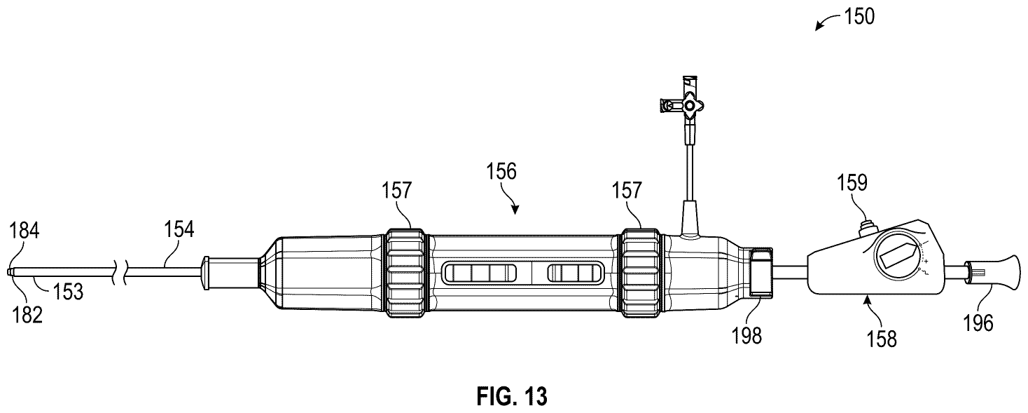

FIG. 13 illustrates the delivery apparatus 150, according to one example. The delivery apparatus 150 can also be referred to as a “dock delivery apparatus,” “dock delivery catheter,” or “dock delivery system.” The delivery apparatus 150 comprises the delivery shaft 154, a handle 156 (which may also be referred to as a “dock delivery system handle”) coupled to a proximal end portion of the delivery shaft 154, a sleeve shaft 182 configured to extend through the delivery shaft 154 and the handle 156, a hub assembly 158 (which may also be referred to as a “dock handle”) coupled to a proximal end portion of the sleeve shaft 182, the pusher shaft 184 configured to extend through the handle 156 and the sleeve shaft 182, and a sleeve handle 196 coupled to a proximal end portion of the sleeve shaft 182.

The delivery shaft 154, which in some examples can be similar to the delivery shaft 54, is configured to be advanced through the patient's vasculature (blood vessel 12) and to the implantation site (such as native mitral valve 16) by the user and may be configured to retain the docking device 152 in a distal end portion 153 of the delivery shaft 154. During the docking device implantation procedure, the delivery shaft 154 is advanced through the catheter shaft 34 of the guide catheter 30 (for example, through a central lumen thereof, etc.) and to the target implantation site.

The handle 156, which in some examples can be similar to the handle 56, is configured to be gripped and/or otherwise held by the user, outside the body of the patient 10, to advance the delivery shaft 154 through the patient's vasculature (such as the blood vessel 12). In some examples, the handle 156 can comprise one or more articulation members 157 (such as rotatable knobs) that are configured to aid in navigating the delivery shaft 154 through the blood vessel 12 by steering or controlling the flexing of the delivery apparatus 150 (for example, the delivery shaft 154, etc.). Some examples of the articulation members 157 can be similar to the articulation members 57. The handle 156 comprises a handle lumen extending through the length of the handle 156, wherein the sleeve shaft 182 and the pusher shaft 184 are configured to be disposed within the handle lumen. Since the sleeve and pusher shafts 182, 184 extending through the handle lumen also extend through the delivery shaft 154, the handle lumen can be coaxially aligned with the delivery shaft 154. In some examples, the handle 156 can further comprise a locking assembly 198 configured to lock a device (for example, the sleeve shaft 182) inserted through the handle lumen, such that the device is selectively prevented from moving relative to the handle 156 of the delivery apparatus 150. In some examples, the locking assembly 198 can be disposed on a proximal end portion of the handle 156.

The hub assembly 158 is configured to be gripped and/or otherwise held by the user, outside the body of the patient 10, to advance the pusher shaft 184 through the patient's vasculature. A distal end portion of the hub assembly 158 is coupled to a proximal end portion of the pusher shaft 184. The axial position of the pusher shaft 184 is controlled by moving the hub assembly 158 in the axial direction relative to the handle 156 and/or the sleeve handle 196. The hub assembly 158 is disposed proximally closer to the user relative to the handle 156, but distally further from the user relative to the sleeve handle 196. The hub assembly 158 comprises a hub assembly lumen extending through the length of the hub assembly 158. The pusher shaft 184 is configured to be disposed within the hub assembly lumen and is coaxial with the sleeve shaft 182 coupled to the distal end portion of the hub assembly 158. In some examples, the hub assembly 158 further comprises a suture lock assembly 159 configured to releasably couple to a proximal end of the release suture 194.

The sleeve handle 196 is configured to be gripped and/or otherwise held by the user, outside the body of the patient 10, to advance the sleeve shaft 182 through the patient's vasculature. The sleeve handle 196 is coupled to a proximal end portion of the sleeve shaft 182 and is disposed proximally closer to the user relative to the handle 156 and the hub assembly 158. The axial position of the sleeve shaft 182 is controlled by moving the sleeve handle 196 in the axial direction relative to the handle 156 and/or the hub assembly 158.

Further details on delivery apparatus/catheters/systems (including various examples of the handle assembly) that are configured to deliver a docking device to a target implantation site can be found in PCT Publication Nos. WO 2020/247907 and WO 2022/072509, and U.S. Patent Publication Nos. 2018/0318079 and 2018/0263764, which are all incorporated by reference herein in their entireties.

Since the variable encircling turn can be adjusted based on relative movement between the pusher shaft 184 and the sleeve shaft 182, the user of the docking device apparatus 150 can adjust the variable encircling turn (as shown in FIGS. 7-8) by moving the pusher shaft 184 in the axial direction relative to the sleeve shaft 182, or vice versa. Since the pusher shaft 184 is coupled to the hub assembly 158 and the sleeve shaft is coupled to the sleeve handle 196, the variable encircling turn can be adjusted in some examples by moving the hub assembly 158 in the distal direction relative to the sleeve handle 196 while the sleeve handle 196 is kept stationary. In some examples, the sleeve handle 196 can be moved in the proximal direction while the hub assembly 158 is kept stationary. In some examples, both the sleeve handle 196 and the hub assembly 158 can be moved in the axial direction. In some examples, the handle 156 can be kept stationary or can be moved relative to at least one of the sleeve handle 196 and the hub assembly 158.

Referring back to FIG. 12, the guide catheter 30 and the delivery apparatus 150 are configured to be coupled to the stabilizer assembly 200, which supports and stabilizes the guide catheter 30 and the delivery apparatus 150 during the procedure. The stabilizer assembly 200 includes a universal platform 202, a stabilizer track 204 mounted to the universal platform 202, one or more supports 206 (for example, clips, clamps, braces, etc.) that can be slidably coupled to the stabilizer track 204, and a hub assembly support 208 that can be slidably coupled to the stabilizer track 204.

The universal platform 202 is a platform configured to support the stabilizer track 204. The universal platform 202 is configured to have an adjustable height and/or orientation, wherein the height and/or orientation can be adjusted relative to a surface on which the universal platform 202 rests (for example, a ground surface or a table surface). In some examples, the universal platform 202 can comprise one or more articulation members 203 (for example, rotatable knobs) for adjusting the height or orientation of the universal platform 202.

The stabilizer track 204 is coupled to a top surface of the universal platform 202. The stabilizer track 204 is configured to be oriented in the axial direction when mounted to the universal platform 202, such that the supports 206 and the hub assembly support 208 can slide along the stabilizer track 204 in the axial direction. In some examples, the stabilizer track 204 can comprise one or more rails 205 extending along the stabilizer track 204 in the axial direction. In the illustrated example, the stabilizer track 204 comprises first and second rail 205a, 205b, but the stabilizer track 204 can comprise one, three, or any suitable number of rails 205. In some examples, each of the rails 205 can comprise an axially extending vertical flange (which is also referred to herein as a “web”) and an axially extending horizontal flange (which is also referred to herein as a “head”) coupled to a topmost end portion of the vertical flange, such that the rails 205 have a “C,” “I”, or “T” shaped cross-section. However, the rails 205 can comprise any suitable cross-section configured to couple to the supports 206 and the hub assembly support 208.

The supports 206 are configured to hold or grip the guide catheter 30 and the handle 156 of the delivery apparatus 150. Each of the supports 206 comprises a post configured to be relocated or repositioned on the stabilizer track 204 in an axial direction, wherein the post is configured to couple to a portion (such as a distal portion) of the guide catheter 30 or the handle 156. In some examples, at least one of the supports 206 can include a position lock 207 configured to prevent the support 206 from moving in an axial direction along the stabilizer track 204. In some examples, the position lock 207 can comprise a threaded shaft that is movable between a locked configuration and an unlocked configuration. When the position lock 207 is in the locked configuration, the threaded shaft comes into frictional contact with the stabilizer track 204, wherein the frictional contact prevents the support 206 from moving in the axial direction relative to the stabilizer track 204.

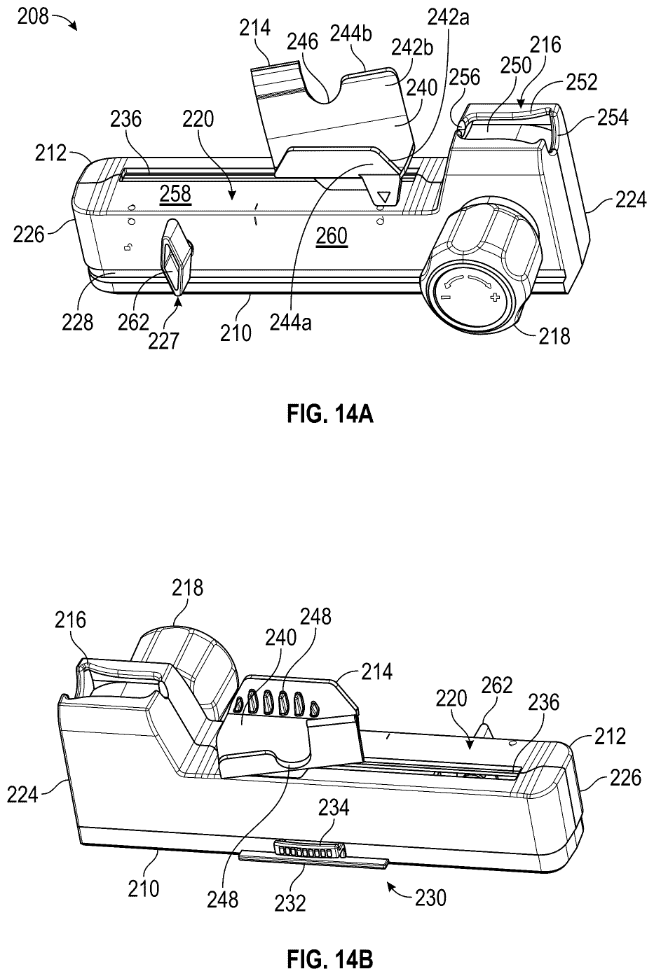

FIGS. 14A-14B illustrate perspective views of the hub assembly support 208, according to one example. The hub assembly support 208 is configured to slidably couple to the stabilizer track 204 and hold or grip the hub assembly 158 and the sleeve handle 196. The hub assembly 158 comprises a base portion 210, a housing 212, a hub assembly cradle 214, a sleeve handle cradle 216, and an actuation control 218. In some examples, the hub assembly support 208 can further comprise an indicator 220 and a brake 227.

The base portion 210 is configured to rest upon and slidingly couple to the stabilizer track 204. In some examples, the base portion 210 can comprise a plate extending from a proximal end portion 224 of the hub assembly support 208 to a distal end portion 226 of the hub assembly support 208.

In some examples, the base portion 210 can further comprise a groove 228 disposed on a first lateral surface of the base portion 210. In some examples, the groove 228 can extend from the proximal end portion 224 to the distal end portion 226 of the base portion 210. The groove 228 can be configured to facilitate the sliding coupling of the base portion 210 to the stabilizer track 204 and to limit vertical movement of the hub assembly support 208. In some examples, the groove 228 can be configured to receive the head of one of the axially extending rails 205 (for example, the first rail 205a).

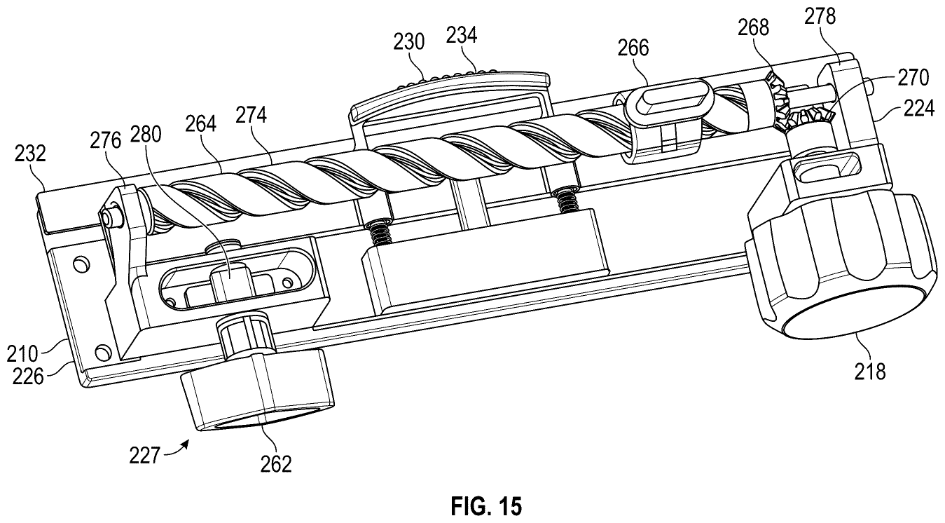

In some examples, the base portion 210 can further comprise a stabilizer track lock 230 configured to slidingly couple the base portion 210 to the stabilizer track 204. The stabilizer track lock 230 can be disposed on a second lateral surface of the base portion 210, wherein the second lateral surface is opposite the first lateral surface. In some examples, the stabilizer track lock 230 can comprise a locking flange 232 that extends from the second lateral surface of the base portion 210 in the lateral direction and a toggle 234 coupled to the locking flange 232. The locking flange 232 can be slidingly coupled to the base portion 210 such that actuating the toggle 234 causes the locking flange 232 to move in the lateral direction between a locked configuration and an unlocked configuration. In the locked configuration, the locking flange 232 can extend laterally outwards from the base portion 210 and underneath the head of the second rail 205b, thereby coupling the base portion 210 to the stabilizer track 204. In the unlocked configuration, the locking flange 232 can at least partially retract within the base portion 210 or the housing 212, thereby permitting the hub assembly support 208 to be decoupled from the stabilizer track 204. In some examples, the stabilizer track lock 230 can be biased in the locked configuration by coupling the laterally extending flange to the base portion 210 via one or more biasing springs (FIG. 15).

The housing 212 is coupled to the base portion 210 and is configured to cover one or more internal components of the hub assembly support 208. In some examples, the housing 212 can be disposed on top of the base portion 210. The housing 212 covers the base portion 210 from the proximal end portion 224 to the distal end portion 226. The housing 212 comprises an axially oriented slot 236 configured to receive a traveler (FIG. 15) that couples the hub assembly cradle 214 to a linear actuator (FIG. 15) disposed within the housing 212. The traveler is also referred to herein as a “carriage.” Although the slot 236 is shown in FIGS. 14A-14B as disposed on a top surface of the housing 212, the slot 236 can alternatively be disposed on one of the lateral surfaces of the housing 212.

In some examples, the base portion 210 and the housing can be formed as a unitary component (such as a chassis).

The hub assembly cradle 214 is configured to receive the hub assembly 158. In some examples, the hub assembly cradle 214 can comprise a laterally extending base flange 240 with a first lateral edge portion 242a and a second lateral edge portion 242b disposed on opposite lateral edges of the base flange 240. The hub assembly cradle 214 can comprise a first lateral flange 244a extending upwards from the first lateral edge portion 242a and a second side flange 244b extending upwards from the second lateral edge portion 242b. In some examples, the curvature of the first and second lateral edge portions 242a, 242b can follow the curvature of the lateral side portions of the hub assembly 158 to ensure that the hub assembly 158 is securely received within the hub assembly cradle 214. In some examples, the base flange 240, at least one of the first lateral flange 244a and the second lateral flange 244b can further comprise a cutout 246 to accommodate a feature of the hub assembly 158 (such as a feature of the suture lock assembly 159).

In some examples, as best shown in FIG. 14B, the hub assembly cradle 214 can further comprise one or more gripping elements 248 disposed on a laterally inward surface of one or more of the lateral flanges 244a, 244b. The gripping elements 222 can be configured to be disposed between one of the lateral flanges 244a, 244b and the hub assembly 158, thereby frictionally secure the hub assembly 158 in the hub assembly cradle 214. The gripping elements 222 can be formed of rubber, polymeric material, or any material with a sufficient coefficient of friction to frictionally engage the hub assembly 158.

The hub assembly cradle 214 is configured to be actuatable in the axial direction relative to the other components of the hub assembly support 208 (such as the base portion 210, the housing 212, and the sleeve handle cradle 216). The hub assembly cradle 214 is coupled to a linear actuator (FIG. 15) disposed within the housing 212, wherein the linear actuator is configured to move the hub assembly cradle 214 in the axial direction along the length of the slot 236. In the illustrated example, a bottom surface of the base flange 240 is coupled to the linear actuator (FIG. 15) via a traveler (FIG. 15) extending through the slot 236 between the base flange 240 and the linear actuator. However, any suitable portion of the hub assembly cradle 214, including the first and second lateral flanges 244a, 244b, can be coupled to the linear actuator and/or the traveler.

The sleeve handle cradle 216 is configured to receive the sleeve handle 196. The sleeve handle cradle 216 is disposed on the housing 212 and is proximally positioned on the housing 212 relative to the hub assembly cradle 214. In some examples, the sleeve handle cradle 216 can comprise one or more cutouts 252, 254, 256 that define a recess 250 configured to receive at least a portion of the sleeve handle 196. In the illustrated example, the sleeve handle cradle 216 comprises a recess 250 defined by a first bell-shaped cutout 252 on a top surface of the housing 212 and a second semicircular cutout 254 on a proximal surface of the housing 212. To better ensure that the sleeve handle 196 is securely received within the recess, the first cutout 252 can have the same shape (for example, a bell shape) as the cross-section of the sleeve handle 196, such that the edge of the first cutout 252 is flush against the surface of the sleeve handle 196. In some examples, the sleeve handle cradle 216 can further comprise a third cutout 256 distally disposed relative to the first cutout 252 and the second cutout 254 configured to accommodate the sleeve shaft 182 which extends from a distal end portion of the sleeve handle 196 towards the hub assembly 158.

In some examples where the sleeve shaft 182 is additionally or alternatively moved relative to the pusher shaft 184 to adjust the variable encircling turn, the sleeve handle cradle 216 can be actuatable relative to the other components of the hub assembly support 208 (such as the base portion 210, the hub assembly cradle 214, etc.). In some examples, the sleeve shaft 182 can additionally or alternatively be coupled to a linear actuator (which can be similar to linear actuator 264).

The actuation control 218 is operatively coupled to the linear actuator (FIG. 15) and is configured to control the degree of actuation of the hub assembly cradle 214 relative to the hub assembly support 208. In some examples, the actuation control 218 can be a rotatable knob. However, the actuation control 218 can be any suitable interface or control (such as a button, a slider, a switch, a crank, etc.) for controlling the linear actuator. In some examples, a user can actuate the hub assembly cradle 214 by grabbing the hub assembly 158 and sliding the hub assembly 158 in the axial direction relative to the other components of the hub assembly support 208.

In some examples, the hub assembly support 208 can further comprise an indicator 220 configured to indicate a magnitude of the variable encircling turn. Since the radius of curvature of the sleeve shaft leading turn 187 correlates to the axial position of the pusher shaft 184 relative to the sleeve shaft 182, and the axial position of the pusher shaft 184 relative to the sleeve shaft 182 correlates to the axial position of the hub assembly cradle 214 relative to the sleeve handle cradle 216, the radius of curvature of the sleeve shaft leading turn 187 can be determined based on the relative axial positions of the hub assembly cradle 214 and the sleeve handle cradle 216. In some examples, the indicator 220 can comprise one or more markings disposed along the length of the slot 236. A first marking 258 disposed towards a proximal end of the slot 236 can indicate that the radius of curvature of the sleeve shaft leading turn 187 (in other words, the variable encircling turn) is equal to the first radius of curvature (r₁) and a second marking 260 disposed towards a distal end of the slot 236 can indicate that the radius of curvature of the sleeve shaft leading turn 187 is equal to the second radius of curvature (r₂).

The brake 227 is configured to restrict the movement of the hub assembly support 208 relative to the stabilizer track 204. In some examples, the brake 227 can comprise a knob 262, a cam (FIG. 15), and a brake pad, wherein the brake 227 can be movable between a locked configuration and an unlocked configuration. When a user actuates the knob 262 to move the brake 227 to the locked configuration, the brake pad can come into frictional engagement with the stabilizer track 204, thereby locking the hub assembly support 208 in a fixed axial position on or relative to the stabilizer track 204.

FIG. 15 illustrates the hub assembly support 208 with the housing 212 and the hub assembly cradle 214 removed. The interior of the hub assembly support 208 comprises a linear actuator 264, a traveler 266 (which is also referred to herein a “carriage”) coupled to the linear actuator 264, and first and second bevel gears 268, 270 operatively coupling the linear actuator 264 to the actuation control 218. In some examples, in which the hub assembly support 208 comprises the stabilizer track lock 230, the hub assembly support 208 can further comprise one or more biasing springs 272.

The linear actuator 264 is configured to actuate the hub assembly cradle 214 relative to the sleeve handle cradle 216, which is disposed in a fixed axial position on the housing 212. The linear actuator 264 is configured to actuate the hub assembly cradle 214 in the axial direction along the length of the slot 236. In some examples, the linear actuator 264 can comprise a threaded shaft 274. The threaded shaft 274 can be coupled a first actuator post 276 and a second actuator post 278 that are axially aligned with each other and coupled to the base portion 210. The threaded shaft 274 can be oriented in the axial direction between the first actuator post 276 and the second actuator post 278. Although the linear actuator 264 is shown in FIG. 15 as the threaded shaft 274, some examples of the linear actuator 264 can comprise hydraulic linear actuators, pneumatic linear actuators, rack and pinion linear actuators, belt-driven linear actuators, or any suitable mechanical or electromechanical linear actuator devices. In some examples, the extent of actuation of the linear actuator 264 can be coterminous with the length of the slot 236 disposed on the housing 212.

The threaded shaft 274 can have a thread pitch defining the distance between adjacent threads on the threaded shaft 274. The speed at which the hub assembly cradle 214 is actuated in the axial direction relative to the rest of the hub assembly support 208 can depend in part on the thread pitch. For example, if the threaded shaft 274 has a relatively high thread pitch, the hub assembly cradle 214 will actuate slower in the axial direction than if the threaded shaft 274 has a relatively low thread pitch. Thus, the thread pitch of the threaded shaft 274 can be selected based in part on a desired actuation speed of the hub assembly cradle 214.

The traveler 266 is configured to extend through the slot 236 to couple the hub assembly cradle 214 to the linear actuator 264. When the threaded shaft 274 is rotated, the rotation can cause the traveler 266 to move proximally or distally in the axial direction along the length of the slot 236, thereby moving the hub assembly cradle 214 in the axial direction as well.

The first and second bevel gears 268, 270 are configured to translate torque between the actuation control 218 and the linear actuator 264. In examples in which the linear actuator 264 comprises the threaded shaft 274, the first bevel gear 268 can be coupled to an end portion (such as a proximal end portion) of the threaded shaft 274. The second bevel gear 270 can be coupled to the actuation control 218. The first bevel gear 268 and the second bevel gear 270 can be disposed at a right angle to each other. Since the first bevel gear 268 and the second bevel gear 270 are in meshed contact with each other, the first bevel gear 268 and the second bevel gear 270 can translate the torque or rotational motion of the actuation control 218 to the threaded shaft 274.

In some examples, in which the hub assembly support 208 comprises the stabilizer track lock 230, the hub assembly support 208 can further comprise one or more biasing springs 272 configured to bias the stabilizer track lock 230 in the locked configuration. The biasing springs 272 can comprise laterally oriented springs disposed between the locking flange 232 or the toggle 234 and the base portion 210. The biasing springs 272 can bias the locking flange 232 in the locked configuration by forcing the locking flange 232 laterally outwards from the base portion 210.

In some examples, in which the hub assembly support 208 comprises the brake 227, the brake 227 can include a cam 280 coupled to the rotatable knob 262 and a brake pad configured to selectively extend through a cutout in the base portion 210. When the knob 262 is rotated to the unlocked configuration, the brake pad can retract within the hub assembly support 208 such that the brake pad does not directly contact the stabilizer track 204. When the knob 262 is rotated to the locked configuration, the cam can engage the brake pad such that the brake pad extends through the cutout in the base portion 210 to frictionally engage or contact the stabilizer track 204. In some examples, the brake 227 can further comprise at least one biasing member to bias the brake pad into contact with the stabilizer track 204. In some examples, the biasing member can comprise a spring. In some examples, the brake pad can be formed from silicone. However, the brake pad can be formed of any material with a sufficient coefficient of friction to frictionally engage the stabilizer track 204.

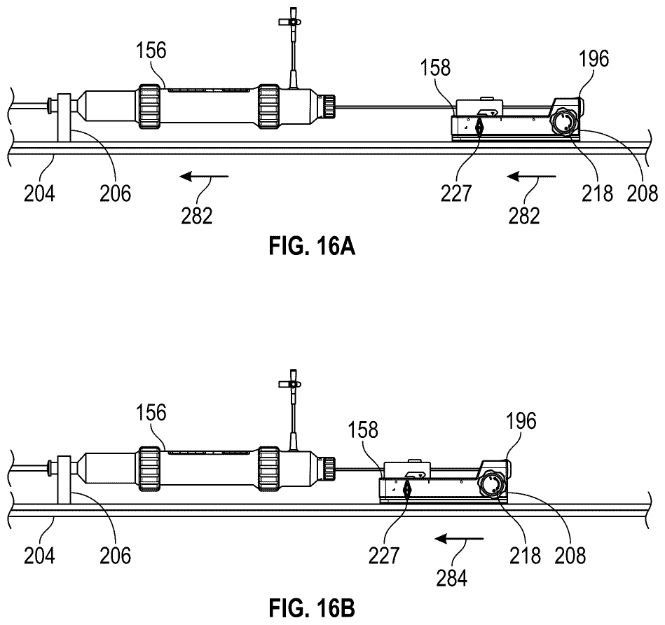

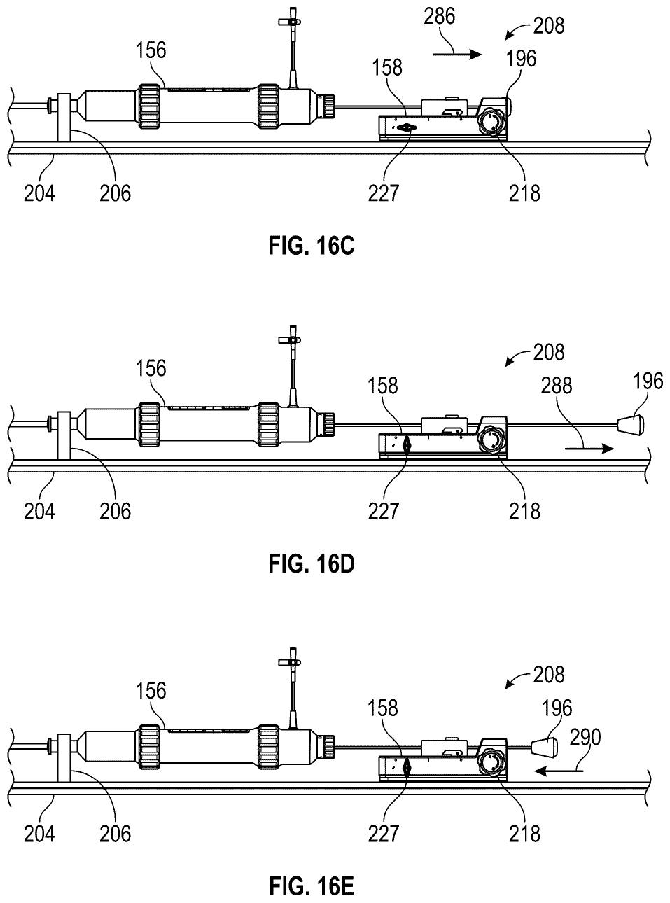

FIGS. 16A-16E illustrate the configuration of the delivery system 100 during the example docking device delivery procedure illustrated in FIGS. 5-11. More specifically, FIGS. 16A-16E illustrate the relative axial positions of the handle 156, the hub assembly 158, the hub assembly support 208, and the sleeve handle 196 during various stages of the example procedure. Although the delivery system 100 can further comprise other components such as the guide catheter 30, these components are omitted from FIGS. 16A-16E for clarity.

FIGS. 16A-16E show the hub assembly 158 as moving relative to the hub assembly support 208 during the adjustment of the variable encircling turn. However, since adjustments to the variable encircling turn are caused by relative movement between the hub assembly 158 and the sleeve handle 196, it should be understood that in some examples either the hub assembly 158, the sleeve handle 196, or both components can be moved relative to each other to adjust variable encircling turn. Furthermore, the handle 156 may be held static or may be moved during various examples of steps for adjusting the variable encircling turn.

FIG. 16A illustrates the configuration of the delivery system 100 during the stage of the example docking device delivery procedure illustrated in FIG. 5. After the guide catheter 30 is deployed, the delivery shaft 154, the sleeve shaft 182, and the pusher shaft 184 are distally advanced in unison out of the guide catheter 30 by advancing the handle 156 in unison with the hub assembly support 208 along the stabilizer track 204 (indicated by arrow 282). In some examples where the hub assembly support 208 comprises the brake 227, the brake 227 is actuated to the unlocked configuration during this stage to allow the hub assembly support 208 to move relative to the stabilizer track 204.

FIG. 16B illustrates the configuration of the delivery system 100 during the stage of the example docking device delivery procedure illustrated in FIGS. 6-7. After the delivery shaft 154 is advanced to the native mitral valve 16, the sleeve shaft 182 and the pusher shaft 184 are advanced in unison out of the delivery shaft 154 by advancing the hub assembly support 208 along the stabilizer track 204 in the distal direction (indicated by arrow 284). The handle 156 remains in a fixed axial position on the stabilizer track 204 while the hub assembly support 208 is distally advanced along the stabilizer track 204 towards the handle 156. In some examples, in which the hub assembly further comprises the brake 227, the brake 227 can be actuated to the unlocked configuration during this stage to allow for relative axial movement between the hub assembly support 208 and the stabilizer track 204.

FIG. 16C illustrates the configuration of the delivery system 100 during the optional stage of the example docking device delivery procedure illustrated in FIG. 8, when the delivery apparatus 150 adjusts the variable encircling turn. During this stage, the user activates the actuation control 218 (for example, by turning the knob), thereby causing the linear actuator 264 to move the hub assembly cradle 214 in the proximal direction (indicated by arrow 286) relative to the sleeve handle cradle 216 to increase the radius of curvature of the sleeve shaft leading turn 187. In some examples, in which the hub assembly support 208 comprises the brake 227, the brake 227 can be actuated to the locked configuration to lock the hub assembly support 208 to the stabilizer track 204, such that the brake pad of the brake 227 frictionally engages the stabilizer track 204. In some examples, the support 206 holding the handle 156 can be locked to the stabilizer track 204 using the position locks 207 to prevent axial movement of the delivery shaft 154 during this stage. However, since the adjustment of the variable encircling turn is caused by relative movement between the hub assembly 158 and the sleeve handle 196, some examples of this stage can involve advancing (in the direction opposite arrow 286) the sleeve handle 196 in the distal direction relative to the hub assembly 158 or moving both the hub assembly 158 and the sleeve handle 196.

FIG. 16D illustrates the configuration of the delivery system 100 during the optional stage of the example docking device delivery procedure illustrated in FIG. 9, when the sleeve shaft 182 is retracted to unsheathe the guard member 180. During this stage, the sleeve handle 196 can be independently actuated by removing or decoupling the sleeve handle 196 from the sleeve handle cradle 216 and moving the sleeve handle 196 in the proximal direction (indicated by arrow 288) relative to the handle 156, the hub assembly 158, and the hub assembly support 208. In some examples, the hub assembly support 208 can be locked to the stabilizer track 204 (for example, using the brake 227) to prevent axial movement of the hub assembly support 208 relative to the stabilizer track 204 during this stage. However, since the guard member 180 is unsheathed due to relative movement between the docking device 152 and the sleeve shaft 182 and/or the handle 156, some examples of this stage can involve advancing (in the opposite direction of arrow 288) the handle 156 and/or the hub assembly 158 in the distal direction relative to the sleeve handle 196 or moving any combination of the handle 156, the hub assembly 158, and the sleeve handle 196.

FIG. 16E illustrates the configuration of the delivery system 100 during the optional stage of the example docking device delivery procedure illustrated in FIG. 10, when guard member 180 is axially foreshortened by advancing the distal end portion 186 of the sleeve shaft 182 in the distal direction (indicated by arrow 290) to exert a force against the proximal end portion 193 of the guard member 180. In some examples, when the proximal end portion 193 is advanced in the distal direction, the proximal end portion 193 can frictionally engage the docking device 152 after the guard member 180 is axially foreshortened such that the guard member 180 is retained in its deployed configuration after the sleeve shaft 182 is removed from the patient's vasculature. However, since the guard member 180 axially foreshortens due to relative movement between the docking device 152 and the sleeve shaft 182, some examples of this stage can involve retracting (in the opposite direction of arrow 290) the hub assembly 158 in the proximal direction relative to the sleeve handle 196 or moving both the hub assembly 158 and the sleeve handle 196.

In some examples, the support 206 holding the handle 156 can be locked to the stabilizer track 204 using the position locks 207 to prevent the delivery shaft 154 from moving during this stage. However, since the movements illustrated in FIGS. 16A-16E are relative movements, the handle 156 can move relative to at least one of the hub assembly 158 and the sleeve handle 196 in any of the illustrated stages. In some examples, the hub assembly support 208 can be locked to the stabilizer track 204 (for example, using the brake 227).

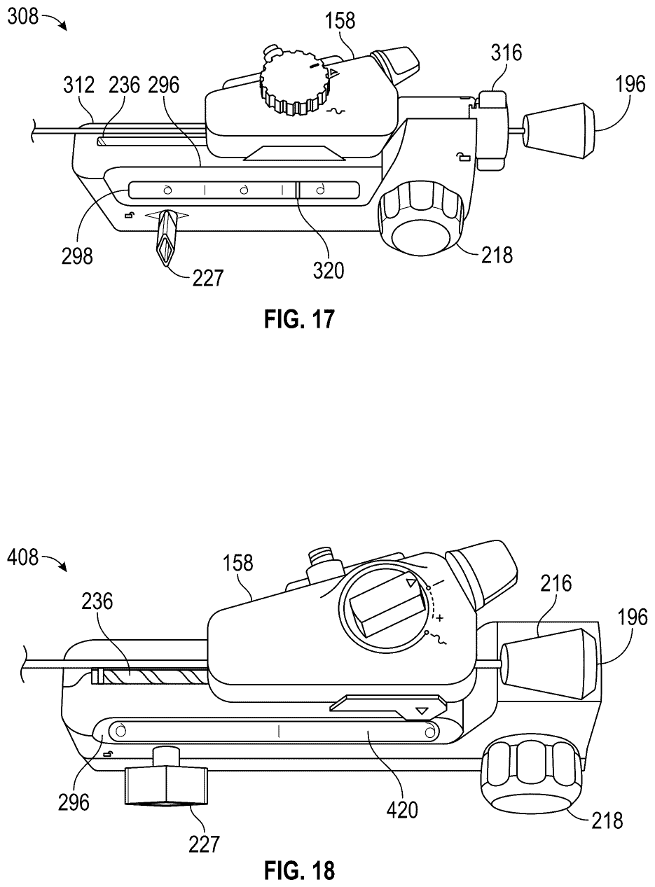

FIG. 17 illustrates a hub assembly support 308, according to a second example. The hub assembly support 308 is depicted with the hub assembly 158 received within the hub assembly cradle 214. One exemplary difference between the hub assembly support 208 of FIG. 14A-14B and the hub assembly support 308 of FIG. 17 is that the hub assembly support 308 can comprise a cam lock 316 instead of the sleeve handle cradle 216 for securing the sleeve handle 196. The cam lock 316 is actuatable between a locked configuration and an unlocked configuration. When the cam lock 316 is in the locked configuration, the cam lock 316 can frictionally contact the sleeve shaft 182, thereby preventing the sleeve handle 196 coupled to the sleeve shaft 182 from moving in the axial position relative to the hub assembly cradle 214. When the cam lock 316 is in the unlocked configuration, the sleeve handle 196 can be free to move in the axial direction relative to the hub assembly cradle 214 (for example, during the stages of the example docking device delivery procedure illustrated in FIGS. 16D-16E).

A second exemplary difference between the hub assembly support 208 of FIG. 14A-14B and the hub assembly support 308 of FIG. 17 is that the hub assembly support 308 can comprise a housing 312 with a chamfer 296. The chamfer 296 can comprise a viewport 298 that extends in the axial direction along the length of the slot 236. An indicator 320 that is visible to the user can be disposed behind the viewport 298. The indicator 320 can comprise a rod coupled to the traveler 266. Since the indicator 320 is coupled to the traveler 266, and the hub assembly is indirectly coupled to the traveler 266, the axial position of the indicator 320 can correlate to the axial positions of the hub assembly 158 and the pusher shaft 184. Thus, as the hub assembly cradle 214 is actuated along the length of the slot 236, the indicator 320 can align with one or more markings disposed on the viewport 298 to indicate the radius of curvature of the sleeve shaft leading turn 187 determined based on the axial position of the hub assembly cradle 214.

FIG. 18 is a hub assembly support 408, according to a third example. One exemplary difference between the hub assembly support 408 and the previously illustrated hub assembly supports 208, 308 is that the hub assembly support 408 can comprise the sleeve handle cradle 216 and the housing 312 with the chamfer 296.

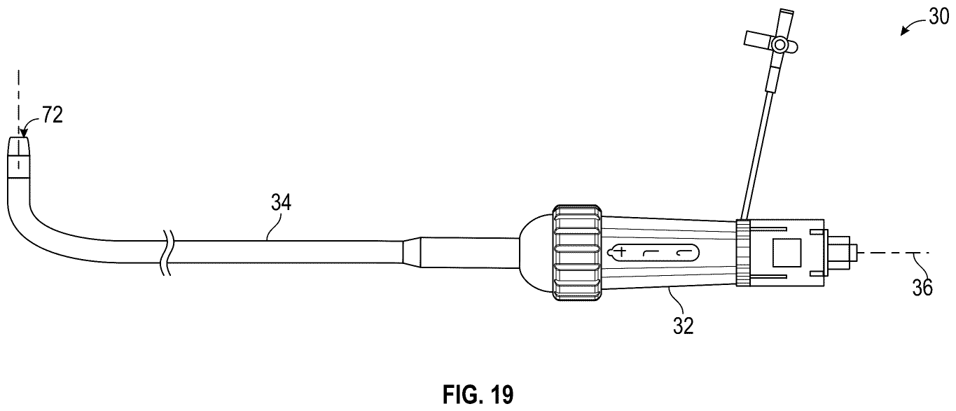

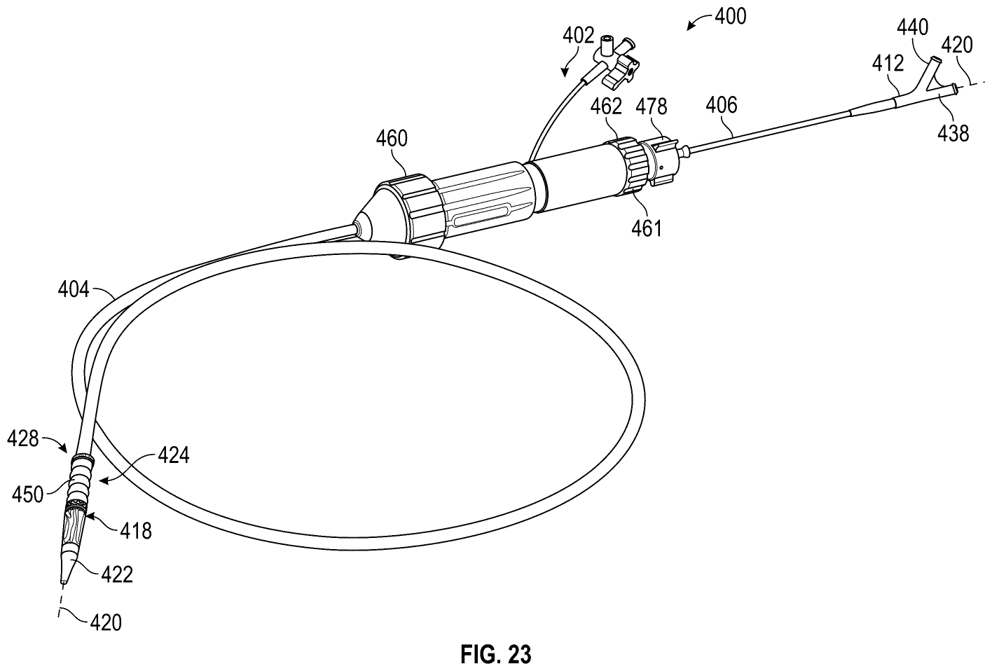

FIG. 19 illustrates the guide catheter 30 (which may be referred to herein as an “introducer device”), according to one example. In some examples, the guide catheter 30 can be used in a prosthetic valve implantation procedure, as described above with reference to FIGS. 1-4. In some examples, the guide catheter 30 can be used in a docking device implantation procedure, as described with reference to FIGS. 5-11. The guide catheter 30 is be configured to be inserted into a patient's vasculature and receive an implant catheter (and/or other delivery apparatus) therein to introduce the implant catheter into the patient's vasculature and at least partially guide the implant catheter to a target implantation site. Examples of implant catheters for prosthetic medical devices (referred to below as “delivery apparatus 150” and “delivery apparatus 400”) that can be received within the guide catheter 30 are shown in FIGS. 13 and 23 respectively.

The guide catheter 30 in the illustrated example comprises the handle 32, the catheter shaft 34 extending distally from the handle 32, and a longitudinal axis 36. In some examples, the catheter shaft 34 can extend proximally into the handle 32. In some examples, the catheter shaft 34 can be coupled to a distal end portion of the handle 32. The handle 32 comprises a catheter handle lumen (not pictured) extending through the length of the handle 32. The catheter handle lumen is axially aligned with the catheter shaft lumen and coupled to a distal end portion of the catheter shaft 34, such that the delivery shaft 154, the sleeve shaft 182, and the pusher shaft 184 can extend through the catheter handle lumen and the catheter shaft lumen. In some examples, the catheter shaft lumen and the delivery shaft 154 can be aligned with the longitudinal axis 36.

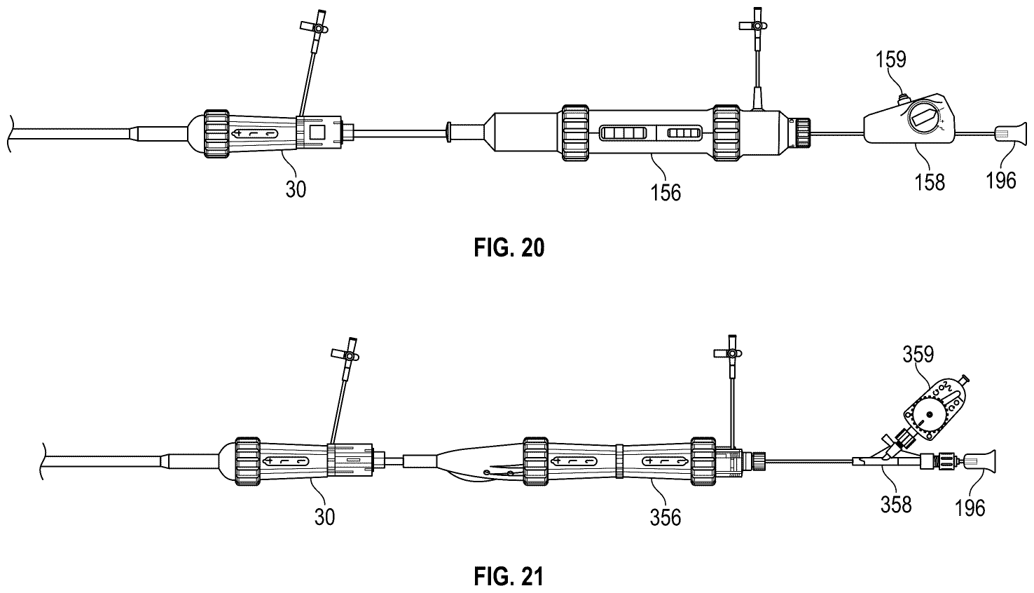

FIG. 20 illustrates the guide catheter 30 coupled to the delivery apparatus 150, according to one example.

FIG. 21 illustrates the guide catheter 30 coupled to a docking device delivery apparatus 350, according to a second example. The docking device delivery apparatus 350 can comprise a handle 356 (which can be similar to the handle 156), a hub assembly 358 (which can be similar to the hub assembly 158) comprising a suture lock assembly 359, and the sleeve handle 196.

FIG. 22 illustrates the docking device 152, according to one example. As depicted in FIG. 22, the docking device 152 in its deployed coiled configuration is configured to receive and secure a prosthetic valve (such as the prosthetic heart valve 62) within the docking device 152, thereby securing the prosthetic valve at the annulus of the native mitral valve 16.

The docking device 152 comprises a coil 188. In some examples, the coil 188 can include a shape memory material (for example, nickel titanium alloy or “Nitinol”) such that the docking device 152 (and the coil 188) can move from a substantially straight configuration (or delivery configuration) when disposed within the delivery shaft 154 to a helical, deployed configuration after being removed from the delivery shaft 154.

The coil 188 has a proximal end 188p and a distal end 188d (which also respectively define the proximal and distal ends of the docking device 152). When being disposed within the delivery shaft 154 (for example, during delivery of the docking device 152 into the vasculature of a patient), a body of the coil 188 between the proximal end 188p and distal end 188d can form a generally straight delivery configuration (without any coiled or looped portions, but can be flexed or bent) so as to maintain a small radial profile when moving through a patient's vasculature. After being removed from the delivery shaft 154 and deployed at an implant position, the coil 188 can move from the delivery configuration to the helical deployed configuration and wrap around native tissue adjacent the implant position. For example, when implanting the docking device at the location of a native valve, the coil 188 can be configured to surround native leaflets of the native valve (and the chordae tendineae that connects native leaflets to adjacent papillary muscles).

The coil 188 in the deployed coiled configuration can include the docking device leading turn 189, the central region 190, and a stabilization turn 195 (or “stabilization coil”) around a central longitudinal axis.

In the deployed coiled configuration, the central region 190 comprises one or more helical turns formed around a central longitudinal axis of the docking device 152, wherein the helical turns have substantially equal radii of curvature configured to encircle the leaflets 24 of the native mitral valve 16. The docking device leading turn 189 extends from a distal end of the central region 190 and has a radius of curvature greater than the radius of curvature of the helical turns of the central region 190. In some examples, the radius of curvature of the docking device leading turn 189 of the docking device 152 is equal to a second radius of curvature, wherein the second radius of curvature is less than the first radius of curvature of the sleeve shaft leading turn 187.

The stabilization turn 195 can extend from a proximal end of the central region 190 and has a diameter greater than the diameter of the central region 190, in the illustrated example. Alternatively, the stabilization turn 195 can have a diameter that is equal, approximately equal, or less than the diameter of the central region 190 (as opposed to larger), and/or the stabilization turn can comprise less of a full turn than depicted in FIG. 22.

In some examples, the docking device 152 can further comprise the guard member 180 disposed on the coil 188. The guard member is configured to reduce the possibility of paravalvular leakage between the native mitral valve 16 and the prosthetic heart valve. In some examples, the guard member 180 can comprise a braided portion disposed between the distal end portion 191 and the proximal end portion 193 of the guard member 180. The braided portion is configured to foreshorten into a deployed configuration when the proximal end portion 193 is forced in a distal direction, wherein the braided portion has an increased radial thickness in the foreshortened, deployed configuration.

Further details of the docking device and its variants are described in PCT Publication No. WO2022/087336, which is incorporated by reference herein in its entirety.

FIG. 23 illustrates a delivery apparatus 400 (which can also be referred to here as an “implant catheter” and/or a “prosthetic heart valve delivery apparatus”) that can be used to implant an expandable prosthetic heart valve, according to one example. In some examples, the delivery apparatus 400 is specifically adapted for use in introducing a prosthetic heart valve into a heart. For example, the delivery apparatus 400 can be used as the prosthetic valve delivery apparatus 60 in a prosthetic valve implantation procedure, as described above with reference to FIG. 3A.

The delivery apparatus 400 in the illustrated example of FIG. 23 is a balloon catheter comprising a handle 402 and a steerable, outer shaft 404 extending distally from the handle 402. The delivery apparatus 400 can further comprise an intermediate shaft 406 (which also may be referred to as a balloon shaft) that extends proximally from the handle 402 and distally from the handle 402, the portion extending distally from the handle 402 also extending coaxially through the outer shaft 404. In some examples, the delivery apparatus 400 can further comprise an inner shaft extending distally from the handle 402 coaxially through the intermediate shaft 406 and the outer shaft 404 and proximally from the handle 402 coaxially through the intermediate shaft.

The outer shaft 404 and the intermediate shaft 406 can be configured to translate (for example, move) longitudinally, along a central longitudinal axis 420 of the delivery apparatus 400, relative to one another to facilitate delivery and positioning of a prosthetic valve at an implantation site in a patient's body.

The intermediate shaft 406 can include a proximal end portion that extends proximally from a proximal end of the handle 402, to an adaptor 412. The adaptor 412 can include a first port 438 configured to receive a guidewire therethrough and a second port 440 configured to receive fluid (for example, inflation fluid) from a fluid source. The second port 440 can be fluidly coupled to an inner lumen of the intermediate shaft 406.

In some examples, the intermediate shaft 406 can further include a distal end portion that extends distally beyond a distal end of the outer shaft 404 when a distal end of the outer shaft 404 is positioned away from an inflatable balloon 418 of the delivery apparatus 400. A distal end portion of the inner shaft can extend distally beyond the distal end portion of the intermediate shaft 406 toward or to a nose cone 422 at a distal end of the delivery apparatus 400.

In some examples, a distal end of the balloon 418 can be coupled to a distal end of the delivery apparatus 400, such as to the nose cone 422 (as shown in FIG. 23), or to an alternate component at the distal end of the delivery apparatus 400 (for example, a distal shoulder). An intermediate portion of the balloon 418 can overlay a valve mounting portion 424 of a distal end portion of the delivery apparatus 400 and a distal end portion of the balloon 418 can overly a distal shoulder of the delivery apparatus 400. As shown in FIG. 23, a prosthetic heart valve 450 can be mounted around the balloon 418, at the valve mounting portion 424 of the delivery apparatus 400, in a radially compressed state. The prosthetic heart valve 450 can be configured to be radially expanded by inflation of the balloon 418 at a native valve annulus, as described above with reference to FIG. 3A.

A balloon shoulder assembly of the delivery apparatus 400, which includes the distal shoulder, is configured to maintain the prosthetic heart valve 450 (or other medical device) at a fixed position on the balloon 418 during delivery through the patient's vasculature.

The outer shaft 404 can include a distal tip portion 428 mounted on its distal end. In some examples, the outer shaft 404 and the intermediate shaft 406 can be translated axially relative to one another to position the distal tip portion 428 adjacent to a proximal end of the valve mounting portion 424, when the prosthetic valve 450 is mounted in the radially compressed state on the valve mounting portion 424 (as shown in FIG. 23) and during delivery of the prosthetic valve to the target implantation site. As such, the distal tip portion 428 can be configured to resist movement of the prosthetic valve 450 relative to the balloon 418 proximally, in the axial direction, relative to the balloon 418, when the distal tip portion 428 is arranged adjacent to a proximal side of the valve mounting portion 424.

An annular space can be defined between an outer surface of the inner shaft and an inner surface of the intermediate shaft 406 and can be configured to receive fluid from a fluid source via the second port 440 of the adaptor 412. The annular space can be fluidly coupled to a fluid passageway formed between the outer surface of the distal end portion of the inner shaft and an inner surface of the balloon 418. As such, fluid from the fluid source can flow to the fluid passageway from the annular space to inflate the balloon 418 and radially expand and deploy the prosthetic valve 450.

An inner lumen of the inner shaft can be configured to receive a guidewire therethrough, for navigating the distal end portion of the delivery apparatus 400 to the target implantation site.

The handle 402 can include a steering mechanism configured to adjust the curvature of the distal end portion of the delivery apparatus 400. In the illustrated example, for example, the handle 402 includes an adjustment member, such as the illustrated rotatable knob 460, which in turn is operatively coupled to the proximal end portion of a pull wire. The pull wire can extend distally from the handle 402 through the outer shaft 404 and has a distal end portion affixed to the outer shaft 304 at or near the distal end of the outer shaft 404. Rotating the knob 460 can increase or decrease the tension in the pull wire, thereby adjusting the curvature of the distal end portion of the delivery apparatus 400. Further details on steering or flex mechanisms for the delivery apparatus can be found in U.S. Pat. No. 9,339,384, as previously incorporated by reference above.

The handle 402 can further include an adjustment mechanism 461 including an adjustment member, such as the illustrated rotatable knob 462, and an associated locking mechanism including another adjustment member, configured as a rotatable knob 478. The adjustment mechanism 361 is configured to adjust the axial position of the intermediate shaft 406 relative to the outer shaft 404 (for example, for fine positioning at the implantation site).

Prosthetic valves disclosed herein (for example, prosthetic heart valve 450, prosthetic heart valve 62, etc.) can be radially compressible and expandable between a radially compressed state and a radially expanded state. Thus, the prosthetic valves can be crimped on or retained by an implant delivery apparatus (for example, delivery apparatus 400, prosthetic valve delivery apparatus 60, etc.) in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

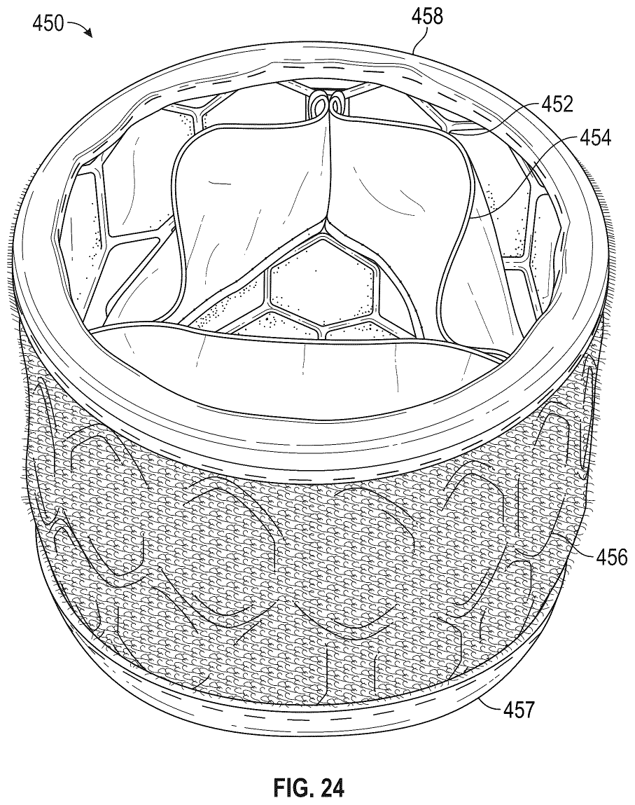

FIG. 24 illustrates the prosthetic valve 450 in a radially expanded position. The prosthetic valve 450 can be used as the prosthetic heart valve 62 in a prosthetic valve implantation procedure, as described above with reference to FIGS. 1-4. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device (for example, docking device 152, etc.) that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

The prosthetic valve 450 can be used as the prosthetic heart valve 62 in a prosthetic valve implantation procedure, as described above with reference to FIGS. 1-4. As shown in FIG. 24, the prosthetic valve 450 can include a frame 452 and a plurality of leaflets 454 can be situated at least partially within the frame 452. The prosthetic valve 450 can also include an outer covering 456 situated about the frame 452. As shown in FIG. 12, the prosthetic valve 450 includes an inflow end 457 and an outflow end 458. The terms “inflow” and “outflow” are related to the normal direction of blood flow (for example, antegrade blood flow) through the prosthetic valve 450. For example, the leaflets 454 can allow blood flow through the valve 450 in a direction from the inflow end 457 to the outflow end 458 and prevent the reverse flow (for example, prevent flow in a direction from the outflow end 458 to the inflow end 457).

The frame 452 can be made of any of various suitable plastically-expandable materials (for example, stainless steel, etc.) or self-expanding materials (for example, Nitinol) as known in the art. When constructed of a plastically-expandable material, the frame 452 (and thus the valve 450) can be crimped to a radially compressed state on a delivery catheter and then expanded inside a patient by an inflatable balloon or equivalent expansion mechanism. When constructed of a self-expandable material, the frame 452 (and thus the valve 450) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 452) include, metal alloys, polymers, or combinations thereof. Example metal alloys can comprise one or more of the following: nickel, cobalt, chromium, molybdenum, titanium, or other biocompatible metal. In some examples, the frame 452 can comprise stainless steel. In some examples, the frame 452 can comprise cobalt-chromium. In some examples, the frame 452 can comprise nickel-cobalt-chromium. In some examples, the frame 452 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The outer covering 456 can be wholly or partly formed of any suitable biological material, synthetic material (for example, any of various polymers), or combinations thereof. In some examples, the outer covering 456 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer covering 456 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU), etc. In some examples, the outer covering 456 can comprise a sponge material or foam, such as polyurethane foam. In some examples, the outer covering 456 can comprise natural tissue, such as pericardium (for example, bovine pericardium, porcine pericardium, equine pericardium, or pericardium from other sources).

Further details of the prosthetic heart valve and its variants are described in U.S. Pat. No. 11,185,406, which is incorporated by reference herein in its entirety.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery system for delivering a prosthetic medical device, the delivery system comprising: a delivery apparatus comprising: a handle; a delivery shaft extending from a distal end portion of the handle and comprising a delivery shaft lumen extending along a length in the delivery shaft; a hub assembly extending from a proximal end portion of the handle; a sleeve shaft disposed within the delivery shaft lumen, the sleeve shaft comprising a sleeve shaft lumen extending along the length of the sleeve shaft; a pusher shaft disposed within the sleeve shaft lumen; a sleeve handle coupled to a proximal end portion of the sleeve shaft; and a hub assembly coupled to a proximal end portion of the pusher shaft; and a stabilizer assembly configured to stabilize the delivery apparatus, the stabilizer assembly comprising: a stabilizer track configured to be oriented in an axial direction; and a hub assembly support configured to slidingly couple to the stabilizer track, comprising: a sleeve handle cradle configured to receive the sleeve handle; a hub assembly cradle configured to receive the hub assembly, wherein the hub assembly cradle is movable in the axial direction relative to the sleeve handle cradle; and a linear actuator coupled to the hub assembly, wherein the linear actuator is configured to actuate the hub assembly cradle in the axial direction relative to the sleeve handle cradle.

Example 2. The delivery system of any example herein, particularly Example 1, wherein delivery system further comprises a guide catheter.

Example 3. The delivery system of any example herein, particularly Example 2, wherein the guide catheter comprises a handle, a catheter shaft extending from a distal end portion of the handle, and a catheter shaft lumen extending along a length of the catheter shaft, wherein the catheter shaft lumen is configured to receive the delivery shaft, the sleeve shaft, and the pusher shaft.

Example 4. The delivery system of any example herein, particularly any one of Examples 2-3, wherein the stabilizer assembly further comprises a support configured to stabilize the guide catheter.

Example 5. The delivery system of any example herein, particularly any one of Examples 1-4, wherein the stabilizer assembly further comprises a support configured to stabilize the handle.

Example 6. The delivery system of any example herein, particularly any one of Examples 1-5, wherein at least one of the sleeve shaft and the pusher shaft have a substantially U-shaped axial cross-section.

Example 7. The delivery system of any example herein, particularly any one of Examples 1-6, wherein the delivery shaft, the sleeve shaft, and the pusher shaft can be independently actuated relative to one another.

Example 8. The delivery system of any example herein, particularly any one of Examples 1-7, wherein the hub assembly support further comprises an indicator.

Example 9. The delivery system of any example herein, particularly Example 8, wherein the indicator is configured to indicate a magnitude of a radius of curvature of a distal end portion of the delivery system.

Example 10. The delivery system of any example herein, particularly Example 9, wherein the radius of curvature of the distal end portion of the delivery system is measured at a sleeve shaft leading turn, wherein the sleeve shaft leading turn is disposed at or adjacent a distal end portion of the sleeve shaft.

Example 11. The delivery system of any example herein, particularly any one of Examples 9-10, wherein the indication of the magnitude of the radius of curvature of the distal end portion of the delivery system is based on a relative axial position of the pusher shaft relative to the sleeve shaft.

Example 12. The delivery system of any example herein, particularly Example 11, wherein the relative axial position of the pusher shaft is based on a relative axial position of the hub assembly cradle relative to the sleeve handle cradle.

Example 13. The delivery system of any example herein, particularly any one of Examples 9-12, wherein the indicator comprises a first marking indicating that the radius of curvature is equal to a first radius of curvature and a second marking indicating that the radius of curvature is equal to a second radius of curvature.

Example 14. The delivery system of any example herein, particularly Example 13, wherein the first marking is disposed in a proximal direction relative to the second marking.

Example 15. A stabilizer assembly configured for use with a delivery apparatus, the stabilizer assembly comprising: a stabilizer track configured to be oriented in an axial direction; and a hub assembly support configured to slidingly couple to the stabilizer track, comprising:
a sleeve handle cradle configured to receive a sleeve handle of the delivery apparatus; a linear actuator configured to move a traveler in an axial direction relative to the sleeve handle cradle; and a hub assembly cradle coupled to the traveler, wherein the hub assembly cradle is configured to receive a hub assembly of the delivery apparatus.

Example 16. The stabilizer assembly of any example herein, particularly Example 15, wherein the hub assembly support further comprises a stabilizer track lock configured to couple the hub assembly support to the stabilizer track, wherein the stabilizer track lock is actuatable between a locked configuration and an unlocked configuration.

Example 17. The stabilizer assembly of any example herein, particularly Example 16, wherein the stabilizer track lock comprises a locking flange extending in a laterally outward direction from the hub assembly support.

Example 18. The stabilizer assembly of any example herein, particularly Example 17, wherein the locking flange is configured to frictionally engage the stabilizer track in the locked configuration.

Example 19. The stabilizer assembly of any example herein, particularly any one of Examples 16-18, wherein the stabilizer track lock is biased in the locked configuration.

Example 20. The stabilizer assembly of any example herein, particularly any one of Examples 16-19, wherein the hub assembly support further comprises a groove disposed on a lateral surface of the hub assembly support.

Example 21. The stabilizer assembly of any example herein, particularly Example 20, wherein the groove is disposed on a first lateral surface of the hub assembly support and the stabilizer track lock is disposed on a second lateral surface of the hub assembly support, wherein the first lateral surface is opposite the second lateral surface.

Example 22. A hub assembly support configured for use with a delivery system, the hub assembly support comprising: a base portion; a housing disposed on the base portion and comprising an axially oriented slot; a sleeve handle cradle disposed on the housing and configured to receive a sleeve handle of the delivery system; a linear actuator coupled to the base portion; a traveler coupled to the linear actuator and extending through the axially oriented slot; and a hub assembly cradle coupled to the traveler, wherein the hub assembly cradle is configured to receive a hub assembly of the delivery system, and wherein the linear actuator is configured to actuate the hub assembly cradle in the axial direction relative to the sleeve handle cradle.

Example 23. The hub assembly support of any example herein, particularly Example 22, wherein the sleeve handle cradle comprises a recess in the base portion configured to receive the sleeve handle.

Example 24. The hub assembly support of any example herein, particularly Example 23, wherein the recess comprises a bell-shaped cutout.

Example 25. The hub assembly support of any example herein, particularly any one of Examples 23-24, wherein the recess comprises a cutout configured to accommodate a sleeve shaft of the delivery system.

Example 26. The hub assembly support of any example herein, particularly any one of Examples 22-25, wherein the hub assembly support further comprises an indicator.

Example 27. The hub assembly support of any example herein, particularly Example 26, wherein the indicator is configured to indicate a magnitude of a radius of curvature of a leading turn of a sleeve shaft coupled to the hub assembly.

Example 28. The hub assembly support of any example herein, particularly Example 27, wherein the indication of the magnitude of the radius of curvature of the leading turn of the sleeve shaft is based on an axial position of the traveler relative to the sleeve handle cradle.

Example 29. The hub assembly support of any example herein, particularly Example 28, wherein the indicator comprises a rod coupled to the traveler.

Example 30. The hub assembly support of any example herein, particularly Example 29, wherein the housing further comprises a viewport extending in the axial direction along a length of the axially oriented slot, and wherein the rod is visible through the viewport.

Example 31. The hub assembly support of any example herein, particularly any one of Examples 29-30, wherein the rod is configured to align with a marking indicating the magnitude of the radius of curvature of the leading turn of the sleeve shaft.

Example 32. A hub assembly support for use with a delivery system, comprising: a base portion; a linear actuator disposed on the base portion, the linear actuator comprising: a threaded shaft oriented in an axial direction; a carriage operatively coupled to the threaded shaft, wherein the linear actuator is configured to actuate the carriage in the axial direction; a hub assembly cradle coupled to the carriage, wherein the hub assembly cradle is configured to receive a hub assembly of the delivery system; and a sleeve handle cradle disposed on the base portion, wherein the sleeve handle cradle is configured to receive a sleeve handle of the delivery system, wherein the linear actuator is configured to actuate the hub assembly cradle relative to the sleeve handle cradle in the axial direction.

Example 33. The hub assembly support of any example herein, particularly Example 32, wherein the hub assembly support further comprises an actuation control configured to control the linear actuator.

Example 34. The hub assembly support of any example herein, particularly Example 33, wherein the actuation control comprises a rotatable knob.

Example 35. The hub assembly support of any example herein, particularly Example 34, wherein the hub assembly support further comprises a plurality of bevel gears operatively coupling the rotatable knob to the threaded shaft.

Example 36. The hub assembly support of any example herein, particularly any one of Examples 32-35, wherein the hub assembly support further comprises a brake configured to restrict movement of the hub assembly support relative to a stabilizer track coupled to the hub assembly support.

Example 37. The hub assembly support of any example herein, particularly any one of Examples 32-36, wherein the extent of actuation of the linear actuator is coterminous with a length of an axially extending slot in the hub assembly support.

Example 38. A method for implanting a prosthetic medical device, comprising: coupling a delivery apparatus to a stabilizer assembly, wherein: the delivery apparatus comprises a delivery shaft, sleeve shaft disposed within the delivery shaft, a pusher shaft disposed within the sleeve shaft, a sleeve handle coupled to a proximal end portion of the sleeve shaft, and a hub assembly coupled to a proximal end portion of the pusher shaft, the stabilizer assembly comprises a hub assembly support comprising a hub assembly cradle configured to receive the hub assembly, a sleeve handle cradle configured to receive the sleeve handle, and a linear actuator configured to actuate the hub assembly cradle in an axial direction relative to the sleeve handle cradle, and coupling the delivery apparatus to the stabilizer assembly comprises coupling the hub assembly into the hub assembly cradle and coupling the sleeve handle to the sleeve handle cradle; advancing the hub assembly support in a distal direction; and actuating the linear actuator to move the hub assembly cradle in an axial direction relative to the sleeve handle cradle.

Example 39. The method of any example herein, particularly Example 38, wherein the prosthetic medical device is a docking device configured for use with a prosthetic heart valve.

Example 40. The method of any example herein, particularly Example 39, wherein the docking device further comprises a guard member.

Example 41. The method of any example herein, particularly Example 40, wherein the method further comprises decoupling the sleeve handle from the sleeve handle cradle, retracting the sleeve handle in a proximal direction relative to the hub assembly, and advancing the sleeve handle in a distal direction relative to the hub assembly.

Example 42. The method of any example herein, particularly any one of Examples 38-41, wherein moving the hub assembly cradle in the axial direction relative to the sleeve handle cradle varies a magnitude of a radius of curvature of a leading turn of a sleeve shaft coupled to the hub assembly.

Example 43. The method of any example herein, particularly any one of Examples 38-42, wherein the linear actuator is configured to move the hub assembly cradle in a distal direction relative to the sleeve handle.

Example 44. The method of any example herein, particularly any one of Examples 38-43, wherein the delivery apparatus further comprises a handle coupled to a proximal end portion of the sleeve shaft, the stabilizer assembly further comprises a support configured to stabilize the handle, and the hub assembly support is advanced in the distal direction in unison with the support.

The features described herein with regard to any example can be combined with other features described in any one or more of the other examples, unless otherwise stated. For example, any one or more of the features of hub assembly support can be combined with any one or more features of another hub assembly support. As another example, any one or more features of one docking device delivery apparatus can be combined with any one or more features of another docking device delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery system for delivering a prosthetic medical device, the delivery system comprising:
a delivery apparatus comprising:
a handle,
a delivery shaft extending from a distal end portion of the handle and comprising a delivery shaft lumen extending along a length in the delivery shaft,
a hub assembly extending from a proximal end portion of the handle,
a sleeve shaft disposed within the delivery shaft lumen, the sleeve shaft comprising a sleeve shaft lumen extending along the length of the sleeve shaft,
a pusher shaft disposed within the sleeve shaft lumen, and
a sleeve handle coupled to a proximal end portion of the sleeve shaft; and
a stabilizer assembly configured to stabilize the delivery apparatus, the stabilizer assembly comprising:
a stabilizer track configured to be oriented in an axial direction, and
a hub assembly support configured to slidingly couple to the stabilizer track, the hub assembly support comprising:
a sleeve handle cradle configured to receive the sleeve handle,
a hub assembly cradle configured to receive the hub assembly, wherein the hub assembly cradle is movable in the axial direction relative to the sleeve handle cradle, and
a linear actuator coupled to the hub assembly, wherein the linear actuator is configured to actuate the hub assembly cradle in the axial direction relative to the sleeve handle cradle.

2. The delivery system of claim 1, wherein the stabilizer assembly further comprises a support configured to stabilize the handle.

3. The delivery system of claim 1, wherein the hub assembly support further comprises a brake configured to lock the hub assembly support to the stabilizer track.

4. The delivery system of claim 1, wherein the hub assembly support further comprises an indicator.

5. The delivery system of claim 4, wherein the indicator is configured to indicate a magnitude of a radius of curvature of a distal end portion of the delivery system.

6. The delivery system of claim 5, wherein the radius of curvature of the distal end portion of the delivery system is measured at a sleeve shaft leading turn, wherein the sleeve shaft leading turn is disposed at or adjacent a distal end portion of the sleeve shaft.

7. The delivery system of claim 4, wherein the indication of the magnitude of the radius of curvature of the distal end portion of the delivery system is based on a relative axial position of the pusher shaft relative to the sleeve shaft.

8. The delivery system of claim 7, wherein the relative axial position of the pusher shaft is based on a relative axial position of the hub assembly cradle relative to the sleeve handle cradle.

9. The delivery system of claim 5, wherein the indicator comprises a first marking indicating that the radius of curvature is equal to a first radius of curvature and a second marking indicating that the radius of curvature is equal to a second radius of curvature.

10. The delivery system of claim 9, wherein the first marking is disposed in a proximal direction relative to the second marking.

11. A stabilizer assembly configured for use with a delivery apparatus, the stabilizer assembly comprising:
a stabilizer track configured to be oriented in an axial direction; and
a hub assembly support configured to slidingly couple to the stabilizer track, comprising:
a sleeve handle cradle configured to receive a sleeve handle of the delivery apparatus;
a linear actuator configured to move a traveler in an axial direction relative to the sleeve handle cradle; and
a hub assembly cradle coupled to the traveler, wherein the hub assembly cradle is configured to receive a hub assembly of the delivery apparatus.

12. The stabilizer assembly of claim 11, wherein the hub assembly support further comprises a stabilizer track lock configured to couple the hub assembly support to the stabilizer track, wherein the stabilizer track lock is actuatable between a locked configuration and an unlocked configuration.

13. The stabilizer assembly of claim 12, wherein the stabilizer track lock comprises a locking flange extending in a laterally outward direction from the hub assembly support.

14. The stabilizer assembly of claim 12, wherein the stabilizer track lock is biased in the locked configuration.

15. The stabilizer assembly of claim 12, wherein the hub assembly support further comprises a groove disposed on a lateral surface of the hub assembly support.

16. The stabilizer assembly of claim 15, wherein the groove is disposed on a first lateral surface of the hub assembly support and the stabilizer track lock is disposed on a second lateral surface of the hub assembly support, wherein the first lateral surface is opposite the second lateral surface.

17. A hub assembly support for use with a delivery system, comprising:
a base portion;
a linear actuator disposed on the base portion, the linear actuator comprising:
a threaded shaft oriented in an axial direction;
a carriage operatively coupled to the threaded shaft, wherein the linear actuator is configured to actuate the carriage in the axial direction;
a hub assembly cradle coupled to the carriage, wherein the hub assembly cradle is configured to receive a hub assembly of the delivery system; and
a sleeve handle cradle disposed on the base portion, wherein the sleeve handle cradle is configured to receive a sleeve handle of the delivery system,
wherein the linear actuator is configured to actuate the hub assembly cradle relative to the sleeve handle cradle in the axial direction.

18. The hub assembly support of claim 17, wherein the hub assembly support further comprises an actuation control configured to control the linear actuator.

19. The hub assembly support of claim 18, wherein the actuation control comprises a rotatable knob.

20. The hub assembly support of claim 19, further comprising a plurality of bevel gears operatively coupling the rotatable knob to the threaded shaft, and a brake configured to restrict movement of the hub assembly support relative to a stabilizer track coupled to the hub assembly support.
